(19) United States
(12) Patent Application Publication
(10) Pub. No.: US 2026/0355449 A1
(43) Pub. Date: Nov. 12, 2026

(54) ACCLIMATISATION FOR RESPIRATORY APPARATUS

(71) Applicant: Fisher & Paykel Healthcare Limited, Auckland (NZ)

(72) Inventors: Robert Stuart KIRTON, Auckland (NZ); Samuel Carey Mathew SANSON, Auckland (NZ); Anton Kim GULLEY, Auckland (NZ); James Alexander Michael REVIE, Auckland (NZ); Frederick Steven THORNBOROUGH, Auckland (NZ)

(21) Appl. No.: 19/139,162

(22) PCT Filed: Dec. 21, 2023
(86) PCT No.: PCT/IB2023/063143

§ 371 Date: Jun. 13, 2025

Related U.S. Application Data
(60) Provisional application No. 63/476,833, filed on Dec. 22, 2022.

(51) Int. Cl. A61M 16/16; A61M 16/00; A61M 16/10

(52) U.S. Cl. CPC A61M 16/161; A61M 16/024; A61M 16/109; A61M 16/1095; A61M 2205/3368; A61M 2205/3653; A61M 2205/505

(57) ABSTRACT

Respiratory apparatus are used in various environments such as hospital or medical facilities to deliver a flow of gases to patients. Improving adherence to respiratory apparatus use improves outcomes for these patients. In one aspect a respiratory apparatus is described comprising a method and flow generator for providing a flow of gas, a humidifier for humidifying the flow of gas and a controller configured to operate the breathing apparatus for acclimatisation by: reducing one or more of a humidity and a temperature of the flow of gas from an operational level to a comfort level, and over a first time period, increasing one or more of the humidity and the temperature of the flow of gas from the comfort level to the operational level.

### FIELD OF THE INVENTION

The present disclosure relates to respiratory apparatus and their method of use to assist with comfort patient.

### BACKGROUND TO THE INVENTION

Respiratory apparatus are used in various environments such as hospital or medical facilities (generally termed “hospital” environment, which covers any facility with trained medical staff) or residential care or home environments (generally termed “home” environment, and may not have trained medical staff) to deliver a flow of gases to patients. A respiratory apparatus generally provides air and/or may be used to deliver supplementary oxygen or other gases with a flow of gases, and/or a humidification apparatus to deliver heated and humidified gases. A respiratory apparatus may allow control over parameters of the gases flow, comprising but not limited to flow rate, pressure, gases concentration, humidity and temperature.

### SUMMARY OF INVENTION

It is an object of the present invention to provide a method and/or apparatus to assist a patient to acclimatise to the gas flow parameters such as humidity and temperature being provided by a respiratory apparatus.

In one aspect the present invention may be said to comprise a respiratory apparatus comprising: a flow generator for providing a flow of gas, a humidifier for humidifying the flow of gas, and a controller configured to operate the breathing apparatus for acclimatisation by: reducing one or more of a humidity and a temperature of the flow of gas from an operational level to a comfort level, and over a first time period, increasing one or more of the humidity and the temperature of the flow of gas from the comfort level to the operational level.

Optionally prior to reducing one or more of the humidity and the temperature of the flow of gas, one or more of the humidity and the temperature of the flow of gas is at an operational level.

Optionally increasing one or more of the humidity and the of the flow of gas comprises ramping one or more of the humidity and the over the first time period.

Optionally a duration of the first time period is determined prior to reducing one or more of the humidity and the of the flow of gases.

Optionally a duration of the time period is: at least about 10 minutes, at least about 15 mins, at least about 30 minutes, between about 30 minutes and about 40 minutes, about 35 minutes, at least about 40 minutes, about 45 minutes, between about 30 minutes and about 50 minutes, between about 30 minutes and about 60 minutes, between about 35 minutes to about 1 hour, at least about 1 hour, between about 1 hour and about 2 hours, or up to about 2 hours, calculated from a rate of about 1 degree C/5 minutes, to ramp from initial comfort level to the operational level (e.g. 31 degrees dew point to 37 degree C dewpoint would take about 30 minutes)

Optionally the time period comprises a maintenance period where the apparatus maintains the initial comfort level, before ramping to the operational level. For example, the maintenance period could be at least about 10 minutes, or at least about 15 minutes or about 30 to 60 minutes. The maintenance period could be instead additional to the ramp time period.

Optionally prior to reducing humidity and/or temperature of the flow of gas, the humidity and/or temperature of the flow of gas is at an operational level for: at least about 10 minutes, at least about 15 mins, at least about 30 minutes, between about 30 minutes and about 40 minutes, about 35 minutes, at least about 40 minutes, about 45 minutes, between about 30 minutes and about 50 minutes, between about 30 minutes and about 60 minutes, between about 35 minutes to about 1 hour, at least about 1 hour, between about 1 hour and about 2 hours, or, up to about 2 hours.

Optionally the operational level of: humidity is about 31, 34 or 37 degrees C dewpoint at 100% RH

Optionally the initial comfort level of: humidity is: 3 degrees below operational dew point level, e.g. 34 degrees dew point when the operational level is 37 degrees dew point.

Optionally one or more of the humidity and the is reduced or increased by controlling the humidifier.

Optionally the humidifier comprises a heater plate configured to heat water to humidify the flow of gas.

Optionally comprising a breathing conduit, the breathing conduit comprising a heater wire. Optionally the heater wire is coupled or couplable to an outlet of the humidifier.

Optionally one or more of the humidity and temperature are reduced or increased by controlling one or more of the heater plate and heater wire.

Optionally the humidity and/or temperature of the flow of gases is at one or more of: an inlet of the humidifier; in the humidifier; an outlet of the humidifier; a breathing conduit; an inlet of the breathing conduit; an outlet of the breathing conduit; a patient interface; an inlet of the patient interface; and an outlet of the patient interface.

Optionally the humidity of the flow of gas is indicated by a humidity parameter. Optionally the temperature of the flow of gas is indicated by a temperature parameter.

Optionally the humidity parameter is one or more of: relative humidity, absolute humidity, dew point.

Optionally the temperature parameter is one or more of: Temperature of gas flow, Temperature of heater plate, Temperature of heater wire.

Optionally the breathing assistance apparatus further comprises one or more humidity and/or temperature sensors at one or more of: the humidifier inlet, in the humidifier, the humidifier outlet, the breathing conduit inlet, in the breathing conduit, the breathing conduit outlet, a patient interface inlet, in the patient interface, a patient interface outlet.

Optionally comprising one or more humidity and/or temperature sensors at one or more of: an inlet of the humidifier; in the humidifier; an outlet of the humidifier; a breathing conduit; an inlet of the breathing conduit; an outlet of the breathing conduit; a patient interface; an inlet of the patient interface; and an outlet of the patient interface.

Optionally the controller is configured to operate the breathing apparatus for patient comfort by upon one or more of: receiving an input from a user, an internal trigger, and start-up of the respiratory apparatus.

Optionally comprising a metering apparatus configured to control a water flow rate in the humidifier.

Optionally one or more of the humidity and temperature are reduced or increased by controlling one or more of the heater plate, heater wire and metering apparatus.

Optionally the step of reducing one or more of a humidity and/or a temperature of the flow of gas from an operational level to a comfort level occurs over a second time period, optionally the second time period being up to 1 hour, up to 30 minutes, up to 20 minutes, between 5 and 15 minutes, or about 10 minutes or less than 5 minutes.

Optionally one or more of the humidity and temperature are ramped ramped over the first time period, optionally the ramp being linear, stepped or non-linear.

Optionally the ramp rate is predefined.

Optionally the humidifier comprises a heated pass-over humidifier, a heated pass-over humidifier with a float, or a vaporization humidifier.

Optionally comprising the step of determining if a user has removed a patient interface and, based on determining removal of a patient interface, determining whether to operate the acclimatisation. Optionally wherein determining whether to operate the acclimatisation comprises determining one or more of the number of times, or length of time the patient interface has been removed.

Optionally the comfort level comprises a dew point between 30% and 75% lower than the operational level.

Optionally one or more acclimatisation actions are used to increase or decrease one or more of the humidity and the temperature of the flow of gas.

Optionally the apparatus comprises a user interface, the user interface configured to allow the user to trigger acclimatisation. Optionally wherein the user interface comprises a button or touch screen.

Optionally comprising a step of selecting each of the one or more acclimatisation actions based on a determination of current patient comfort, optionally patient comfort is determined by one or more of user interface removal and trigger of the acclimatisation button.

Optionally wherein the controller is configured to operate the breathing apparatus for acclimatisation by: reducing a flow rate of the flow of gas from the operational level to the comfort level, and over a first time period, increasing the flow rate of the flow of gas from the comfort level to the operational level.

Optionally reducing the flow rate and increasing the flow rate is simultaneous with reducing one or more of a humidity and a temperature and increasing one or more of a temperature respectively.

Optionally reducing the flow rate and reducing one or more of a humidity and a temperature are performed separately.

Optionally the operational level is a high flow therapy level.

In another aspect the present invention may be said to comprise a breathing assistance apparatus according to claim 1 further comprising: a temperature sensor at the humidifier outlet, wherein the humidity of the flow of gas is indicated by a humidity parameter, the humidity parameter is dew point, such that the comfort level is a first dew point and the operational level is a second dew point, and wherein to increase the humidity from the comfort level to the operational level, the controller controls the apparatus to ramp humidity of the gas flow by: reducing power to the heater plate until the outlet temperature is at or less than the first dew point, and increasing power to heater plate until the outlet temperature is at or above the second dew point.

Optionally the first dew point has a set point in the range of about 30% to about 75%, lower than the second dew point, and optionally in the range of 50 % to 65% lower than the second dew point.

Optionally the first dew point is a set point at least about 3 degrees lower than the second dew point, and more optionally at least about 5 degrees lower than the second dew point and more optionally, wherein the first dew point has a set point of about 27 degrees and the second dew point has a set point of about 37 degrees.

Optionally the time period is in the range of about 15 minutes to 60 minutes, and optionally in the range of about 30 minutes and 45 minutes.

Optionally the power is increased by increasing the duty cycle provided to the heater plate, optionally where the duty cycle ranges from about 10% to 80% over the time period.

Optionally the controller receives input to trigger a ramp mode: from user interface, from an external sensor and/or internal decision that determines patient interface removal and/or non-compliant use, or from start-up.

In another aspect the present invention may be said to comprise a respiratory apparatus further comprising: a temperature sensor at the humidifier outlet and conduit outlet, wherein the humidity and temperature of the flow of gas is indicated by a humidity and temperature parameter, the humidity parameter is dew point, and the comfort level is a first dew point and first temperature and the operational level is a second dew point and second temperature, and wherein to increase the humidity and temperature from the comfort level to the operational level, the controller controls the apparatus to ramp humidity and temperature of the gas flow by: reducing power to the heater plate and heater wire until the humidifier outlet temperature is at or less than the first dew point, and the conduit outlet temperature is at or less than the first temperature, increasing power to heater plate and heater wire until the humidifier outlet temperature and conduit outlet temperature is at or above the second dew point and second temperature.

Optionally the humidity is increased from the first dew point to the second dew point over a first time period.

Optionally the temperature is increased from the first temperature to the second temperature over a second time period.

Optionally the first and second time period are the same.

Optionally the first and second time period are different.

Optionally the first and second temperatures are above the first and second dew points.

In another aspect the present invention may be said to comprise a respiratory apparatus comprising: a flow generator for providing a flow of gas, a humidifier for humidifying a flow of gas, a user interface for interaction by a user a controller in electronic communication with the flow generator and the humidifier, the controller configured to control the operation of the flow generator to generate a flow of gas and control the humidifier to humidify the flow of gas, the controller further arranged in electronic communication with the user interface to receive inputs from the user and provide output to the user, the controller configured to activate a comfort mode in response to receiving an input for the acclimatisation mode via the user interface, during the comfort mode the controller further configured to: reduce the humidity and/or temperature of the flow of gas from an operational level to a comfort level, and over a time period, increase the humidity and/or temperature of the flow of gas from the comfort level to the operational level.

Optionally during comfort mode the humidity/temperature is ramped over a time period from the operational level to the comfort level.

Optionally the time period is predefined before the acclimatisation mode is initiated.

Optionally the ramp is a linear ramp over the time period.

In a further aspect the present invention may be said to comprise a respiratory a respiratory apparatus comprising: a flow generator (blower), humidifier in fluid communication with blower, humidifier configured to humidify gases, controller having two modes: a) operational mode b) acclimatisation mode, controller operating by default in operational mode and controller configured to activate acclimatisation mode in response to a user selection, wherein in operational mode the flow generator is controlled to generate a flow rate of gases and humidifier is controlled to humidify gases to an operational humidity level, wherein in acclimatisation or comfort mode the flow generator is unchanged (or generates flow rate same as operational mode) and humidifier is controlled humidify gases to a comfort level/acclimatisation level, wherein the comfort level or humidity is less that the operational mode of humidity.

Optionally acclimatisation mode reduces irritation to the patient due to higher operational humidity and allows patient to adjust to the therapy.

Optionally during acclimatisation mode, humidity is reduced to comfort level and then increased to the operational level over a time period.

Optionally the time period is a predefined time period.

Optionally the time period is user defined time period.

Optionally during acclimatisation mode the humidity is ramped from comfort level to operational level over the time period.

Optionally the ramp of humidity is a linear ramp, which optionally might be a predefined ramp rate.

Optionally humidity is increased from comfort level to operational level in a non-linear trajectory over the time period.

Optionally multiple time periods may be selectable by the user.

Optionally the flow rate is predefined (either user selected or clinician selected).

Optionally the apparatus comprises a conduit in fluid communication with a humidifier, a conduit positioned between humidifier and a patient interface, and the conduit comprises a wall defining a gases lumen and a heater wire in thermal communication with the lumen to heat the gases in the lumen.

In one aspect the present invention may be said to comprise a respiratory apparatus comprising: a flow generator for providing a flow of gas, a humidifier for humidifying the flow of gas, and a controller configured to operate the breathing apparatus for acclimatisation by: reducing a parameter of the flow of gas from an operational level to a comfort level, and over a time period, increasing the parameter of the flow of gas from the comfort level to the operational level. Optionally the parameter comprises one or more of humidity and/or temperature, flow rate, pressure and gases concentration.

In one aspect the present invention may be said to comprise a respiratory apparatus comprising: a flow generator for providing a flow of gas, a humidifier for humidifying the flow of gas, and a controller configured to operate the breathing apparatus for acclimatisation by: providing a humidity and a temperature of the flow of gas at a comfort level, the comfort level comprising one or more of the humidity and the temperature less than an operational level, and over a first time period, increasing one or more of the humidity and the temperature of the flow of gas from the comfort level to the operational level.

Optionally the comfort level is operated for a second time period.

Optionally comprising the step of reducing one or more of the humidity and the temperature of the flow of gas from the operational level to the comfort level prior to providing the comfort level.

Optionally comprising any one or more of the previous features.

In one aspect the present invention may be said to comprise a method of controlling a respiratory apparatus comprising a humidifier comprising the steps of: providing a humidity and a temperature of a flow of gas in a conduit for a patient interface at a comfort level, the comfort level comprising one or more of the humidity and the temperature less than an operational level, and over a first time period, increasing one or more of the humidity and the temperature of the flow of gas from the comfort level to the operational level.

Optionally prior to reducing one or more of the humidity and temperature of the flow of gas, one or more of the humidity and temperature of the flow of gas is at an operational level.

Optionally increasing one or more of the humidity and temperature of the flow of gas comprises ramping one or more of the humidity and temperature over the first time period.

Optionally a duration of the first time period is determined prior to reducing the humidity and/or temperature of the flow of gases.

Optionally a duration of the first time period is: at least about 10 minutes, at least about 15 mins, at least about 30 minutes, between about 30 minutes and about 40 minutes, about 35 minutes, at least about 40 minutes, about 45 minutes, between about 30 minutes and about 50 minutes, between about 30 minutes and about 60 minutes, between about 35 minutes to about 1 hour, at least about 1 hour, between about 1 hour and about 2 hours, or up to about 2 hours, calculated from a rate of about 1 degree C/5 minutes, to ramp from initial comfort level to the operational level (e.g. 31 degrees dew point to 37 degree C dewpoint would take about 30 minutes)

Optionally prior to reducing one or more of the humidity and temperature of the flow of gas, one or more of the humidity and temperature of the flow of gas is at an operational level for: at least about 10 minutes, at least about 15 mins, at least about 30 minutes, between about 30 minutes and about 40 minutes, about 35 minutes, at least about 40 minutes, about 45 minutes, between about 30 minutes and about 50 minutes, between about 30 minutes and about 60 minutes, between about 35 minutes to about 1 hour, at least about 1 hour, between about 1 hour and about 2 hours, or up to about 2 hours.

Optionally the operational level of humidity is about 31, 34 or 37 degrees C dewpoint at 100% RH.

Optionally the comfort level comprises a humidity 3 degrees below operational dew point level.

Optionally one or more of the humidity and temperature are reduced or increased by controlling the humidifier.

Optionally the humidifier comprises a heater plate configured to heat water to humidify the flow of gas.

Optionally one or more of the humidity and temperature are reduced or increased by controlling one or more of the heater plate and heater wire in a breathing conduit.

Optionally one or more of the humidity and the temperature of the flow of gases is sensed at one or more of: an inlet of the humidifier; in the humidifier; an outlet of the humidifier; a breathing conduit; an inlet of the breathing conduit; an outlet of the breathing conduit; a patient interface; an inlet of the patient interface; and an outlet of the patient interface.

Optionally the humidity of the flow of gas is indicated by a humidity parameter, and the temperature of the flow of gas is indicated by a temperature parameter.

Optionally the humidity parameter is one or more of: relative humidity; absolute humidity; and dew point.

Optionally the temperature parameter is one or more of: Temperature of gas flow; Temperature of heater plate; and Temperature of heater wire.

Optionally the method comprises receiving an indication to begin acclimatisation. Optionally the indication comprises one or more of: receiving input from a user; an internal trigger; and start-up of the respiratory apparatus.

Optionally comprising a metering apparatus configured to control a water flow rate in the humidifier.

Optionally one or more of the humidity and temperature are reduced or increased by controlling one or more of the heater plate, heater wire and metering apparatus of the humidifier.

Optionally the step of reducing one or more of a humidity and/or a temperature of the flow of gas from an operational level to a comfort level occurs over a second time period, optionally the second time period being up to 1 hour, up to 30 minutes, up to 20 minutes, between 5 and 15 minutes, or about 10 minutes or less than 5 minutes.

Optionally one or more of the humidity and temperature are ramped over the first time period, optionally the ramp being linear, stepped or non-linear. Optionally the ramp rate is predefined.

Optionally the humidifier comprises a heated pass-over humidifier, a heated pass-over humidifier with a float, or a vaporization humidifier.

Optionally comprising the step of determining if a user has removed a patient interface and, based on determining removal of a patient interface, determining whether to operate the acclimatisation, optionally wherein determining whether to operate the acclimatisation comprises determining one or more of the number of times, or length of time the patient interface has been removed.

Optionally the comfort level comprises a dew point between 30% and 75% lower than the operational level.

Optionally one or more acclimatisation actions are used to increase or decrease one or more of the humidity and the temperature of the flow of gas.

Optionally comprising a step of selecting each of the one or more acclimatisation actions based on a determination of current patient comfort, optionally patient comfort is determined by one or more of user interface removal and trigger of the acclimatisation button.

Optionally comprising the steps of: reducing a flow rate of the flow of gas from the operational level to the comfort level, and over a first time period, increasing the flow rate of the flow of gas from the comfort level to the operational level.

Optionally reducing the flow rate and increasing the flow rate is simultaneous with reducing one or more of a humidity and a temperature and increasing one or more of a temperature respectively.

Optionally reducing the flow rate and reducing one or more of a humidity and a temperature are performed separately.

Optionally the operational level is a high flow therapy level.

The term “non-sealing patient interface” (i.e., unsealed patient interface) as used herein can refer to an interface providing a pneumatic link between an airway of a patient and a gases flow source (such as from flow generator 11) that does not completely occlude the airway of the patient. A non-sealed pneumatic link can comprise an occlusion of less than about 95% of the airway of the patient. The non-sealed pneumatic link can comprise an occlusion of less than about 90% of the airway of the patient. The non-sealed pneumatic link can comprise an occlusion of between about 40% and about 80% of the airway of the patient. The airway can include one or both nares of the patient and/or their mouth. For a nasal cannula the airway is through the nares. In some configurations, the “non-sealing patient interface” may comprise a tracheal interface.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7)

### BRIEF DESCRIPTION OF DRAWINGS

Examples will be described with reference to the following drawings, of which:

FIG. 1 is a block diagram of a respiratory apparatus that can implement the present examples.

FIG. 2 is a flow diagram of a general example of a method to acclimatise a patient to operational level of flow therapy, which can be implemented on the respiratory apparatus of FIG. 1.

FIG. 3 is a flow diagram of an alternative of any example in which trigger can take place by a respiratory apparatus detecting discomfort in a patient and providing the option of a comfort button for a patient to manually trigger acclimatisation, which can be implemented on the respiratory apparatus of FIG. 1.

FIG. 4 is a flow diagram of a first example of a method to acclimatise a patient to operational level of flow therapy, which can be implemented on the respiratory apparatus of FIG. 1.

FIG. 5 is a flow diagram of a first example of a method to acclimatise a patient to operational level of flow therapy, which can be implemented on the respiratory apparatus of FIG. 1.

FIG. 6 is a plot of an example variation in humidity over the time in minutes from an acclimatisation trigger actuation showing linear changes in dewpoint.

FIG. 7 is a plot of an example variation in humidity and temperature over the time in minutes from an acclimatisation trigger actuation showing linear changes in dewpoint and temperature.

FIG. 8 is a plot of an example variation in humidity over the time in minutes from an acclimatisation trigger actuation showing stepped changes in dewpoint.

FIG. 9 is a plot of an example variation in humidity over the time in minutes from an acclimatisation trigger actuation showing non-linear changes in dewpoint.

FIG. 10 is a block diagram of an alternative respiratory apparatus that can implement the present examples.

FIG. 11 is a flow diagram of an example of a method to acclimatise a patient to operational level of flow therapy, which can be implemented on the respiratory apparatus of FIG. 10.

### DETAILED DESCRIPTION

It should be noted that the present specification refers to humidity. There are various parameters that can directly or indirectly indicate humidity, such as relative humidity, absolute humidity and dew point. These can all be interchangeably converted using relevant conversion parameters. For example, dew point can be converted to a relative humidity if the relevant air pressure and temperature are known. As such, any reference to humidity refers to the concept itself and is not necessarily restricted any particular parameter type that can define or indicate humidity. Also, reference to one type of humidity parameter (e.g. dew point) in an example is not limiting, and the same concept described can be applied using other humidity parameters. The description relates to the control of humidity and should not be limited to the particular parameter used to refer to humidity. But it should also be noted a parameter that can be used to refer to humidity might not actually be a direct measure of humidity and/or might be a different parameter to other parameters that refer to humidity. While each such parameters might be convertible, they are not necessarily directly interchangeable and are different parameters that have their own technical uses. For example, in some circumstances a particular parameter, such as any one or more of dew point, relative humidity and absolute humidity, may be easier to calculate or more suited to a particular method than an alternative parameter of

### Overview

Respiratory apparatus (also termed interchangeably: “respiratory therapy apparatus”, “respiratory support apparatus”, “breathing assistance apparatus”) can provide breathing gas to a patient with controlled flow and/or pressure to support respiratory function. This could be in a hospital or home environment. Typically respiratory apparatus/respiratory function support might comprise (without limitation): High flow therapy (also called nasal high flow, HFT or NHF, e.g. for patients suffering from a lung disease e.g. dyspnea, respiratory distress, pneumonia, emphysema and other obstructive pulmonary diseases), BiLevel/NIV pressure support (including various sub modes e.g. S/T mode, T mode etc.), CPAP support (for OSA), humidifier respiratory therapy, or the like. Such respiratory apparatus can have humidifiers that control humidity of the breathing gas provided to a patient. They can also control the temperature of the breathing gas provided to the patient. Humidity and/or temperature is provided to improve patient experience and health outcomes for the patient. As a non-limiting example, flow therapy uses a respiratory apparatus 1 to provide a gas flow to a patient with a flow rate (e.g. above 15 L/min), temperature (e.g. 37 °C) and a humidity (e.g. a 37 °C dew point or 44 mg of absolute humidity).

When operating, the respiratory apparatus 10 has operational parameters, which can comprise gas flow parameters such as gas flow humidity, temperature, oxygen fraction, pressure and/or flow rate (among others). When providing therapy, the respiratory apparatus 10 has operational parameters that will be at an operational level (that is, a parameter value)—these being parameter levels that provide therapy. Typical parameters values (by way of non-limiting example) are: 37 degrees C dewpoint at 100% RH (relative humidity). Other dewpoint settings could be any one or more of 31, 34, and 37 degrees Celsius.

When the apparatus is being used but is not providing therapy, the operational parameters might be at a non-operational level. Typically, an operational parameter could be described as a therapy parameter, and an operational level could be a specified level, user set level, therapy level or similar depending on the purpose of the operational parameter and/or how it is specified. These might be different or the same levels depending on operational context. Examples are:

Specified level—a specified parameter value specified by a clinician. Typically the specified level will be an operational level, such as a therapy level/prescribed level of the parameter suitable for treating the patient.

User set level—a parameter value set by a user. Typically the user set level will be an operational level, such as a therapy level/prescribed level of the parameter suitable for treating the patient. But it might be some other level and/or it might be specified by the user.

Therapy level—a parameter value (e.g. 37 degrees C with dewpoint at 100% RH. Other dewpoint settings could be 31 degrees Celsius, 34 degrees Celsius) of an operational parameter set to a therapeutic level to provide therapy (such as CPAP, NHF, Bilevel, humidification support) to a patient. It might also be a prescribed level. That is to say the therapy level may be a level set or issued by a clinician that a patient is suggested to use.

Prescribed level—a parameter value of an operational parameter set to a level prescribed by a clinician. It might also be a therapy level.

In general terms, an operational level of an operational parameter is a value provided during operation to provide therapy, irrespective of what it is specified by, or the method or person by which it is set. At other times, an operational parameter might be at a non-operational level that does not provide therapy or does not provide the set or prescribed therapy level. That might include an initial comfort level, as described later.

Compliance to therapy is an important requirement to achieve therapeutic outcomes, particularly for patients using flow therapy at home. Using therapy e.g. high flow therapy for a prescribed time is useful to achieve therapeutic outcomes. A home based patient should use the respiratory apparatus 1 in a prescribed manner (e.g. for a prescribed time per day e.g. at least 4 hours a day, at a prescribed flow rate, humidity, temperature and/or other prescription parameter). A prescription (e.g., a prescribed level of therapy) is provided by a patient's doctor, clinician or the like. Alternatively, patients are encouraged to use the respiratory apparatus when they are feeling breathless or feeling respiratory distress to provide relief to the patient. The patient may use the respiratory apparatus for as long as needed. Generally the longer a patient uses the respiratory apparatus, the better the outcomes for the patient.

However, with a flow of heated, humidified gases some patients can experience discomfort, due to temperature and/or humidity of the breathing gas at the operational level. For example, patients complain of hot/wet or hot/sticky feeling around nose and lips when receiving humidified and heated air. This discomfort can reduce patient compliance with the therapy—e.g. a patient might not use the respiratory apparatus 1 from the prescribed time. This problem is particularly an issue in the home environment as there are no clinicians to provide support. Clinicians want their patients to comply to therapy. Often patients treated at home suffer from a chronic condition e.g. COPD and sufferers are often treated with NHF therapy or BiLevel therapy. Sleep Apnea sufferers are treated with CPAP and compliance is important for this patient group too. That said, compliance is also important for hospital patients, who generally are more acute and suffer from acute respiratory conditions or they may have had an exacerbation. Again compliance and use of the therapy for acute respiratory patients is also very important.

The present disclosure relates to improving patient comfort when using a respiratory apparatus, which in turn results in improved compliance with respiratory apparatus use/therapy. The present disclosure identifies that assisting a patient to acclimatise to (that is, get used to/tolerate) the gas flow (operational) parameters (e.g. temperature and humidity) can improve patient experience (comfort) and therefore compliance. However, patients don't often have any medical staff (e.g. nurses) or other technical staff that can adjust the humidity level and/or temperature to assist.

The present examples provide a respiratory apparatus 10 for providing a gas flow with a flow rate, pressure, oxygen fraction, temperature and humidity (gas flow (operational) parameters), where the humidity and/or temperature gas flow parameters can be controlled (acclimatisation actions) to assist the patient acclimatise to the prescribed humidity and/or temperature, resulting in assisting the patient to achieve compliance.

Acclimatisation of the patient means the humidity/temperature gas flow parameters are changed over time so that the patient does not experience discomfort that might otherwise be experienced if the operational level(s) of the operational parameter(s) are too high for the patient comfort. Rather, due to an acclimatisation method, the patient experiences comfort while the operational parameter(s) are brought up to operational level(s). By comfort, it is meant that as the operational parameters levels are changed over time, the parameter levels are tolerable by the patient at each time until they become used to that level and then the level is changed further until the operational level is achieved. The time over which parameters are changed is slow enough to allow the patient to get used to the parameters. This leads to better compliance outcomes because the patient is comfortable/the provision of gas flow is tolerable. This acclimatisation method might happen e.g. as soon as operational level(s) of the operational parameter(s) are provided, or after they have been provided for some time. The acclimatisation method could also be provided proactively before operational levels are provided, in anticipation that the patient might experience discomfort. It could be triggered at other times too.

As one non-limiting example, an initial comfort level is some humidity and/or temperature level where the patient experiences less discomfort with the heated and humidified gas flow, that is, a level where they can tolerate the temperature, humidity and any other parameter of the gas flow in a manner that they will more likely use the respiratory apparatus. Typically the initial comfort level of the parameter is lower than the operational level of the parameter. The initial comfort level is not the only level of an operational parameter that might be tolerable to a patient but is the one that is used as a starting point. The temperature and/or humidity is increased over a time period to reach the operational level. The temperature and humidity might first also be maintained at the initial comfort level for a maintenance period (which can form part of the time period or be separate to it) before increasing. As the temperature and/or humidity of the gas flow increases from the initial comfort level to the operational level over the time period, the patient can acclimatise to the increasing humidity and/or temperature such that they can tolerate the operational level of the humidity and/or temperature once it is reached. This is because they are exposed to increasing levels of the operational parameter and can acclimatise to each level. The time period is long enough/the ramp is slow enough to allow the patient to get used to the humidity and/or temperature. This means the patient is more likely to use the respiratory apparatus at the operational level, being the prescribed level, and therefore more likely to achieve compliance.

Acclimatisation can be done at any suitable time, such as after the respiratory apparatus has “warmed up”, where during a time period the humidity and/or temperature parameters decrease from an operational level (which could be a prescribed level but could be any other specified level by a clinician or patient that provides therapy) to an initial comfort level, and then over the time period increase backup to the operational level. Prior to increase, the temperature and humidity might first also be maintained at the initial comfort level for a maintenance period. Herein, any reference to raising the temperature and/or humidity over a time period can be deemed to cover maintaining the initial comfort level for a maintenance period a part of the overall time period in which the parameters increase. This gradual increase (that is “ramp” or “ramp rate”) of humidity and/or temperature helps acclimatise the patient to those parameters. The ramp rate could be defined as change in parameter over time, such as change in humidity over time (e.g. change in % RH per minute) or change in temperature over time (e.g. change in degrees C per minute). However, as will be described, acclimatisation can be provided at other times. The time period/ramp rate is long enough/the ramp is slow enough to allow the patient to get used to the humidity and/or temperature. The ramp rate could be linear (constant ramp) or non-linear over time. The ramp rate may be stepped. The ramp rate would be linear after the maintenance period, if there is such a period.

Note, there can be a different time period for varying temperature and for varying humidity. In such cases, there is an overall acclimatisation time period over which all humidity and/or temperature parameters are varied to achieve acclimatisation, but the actual time period over which one of them is varied might be a separate, shorter time period.

### Respiratory Apparatus Examples

There are various examples of a respiratory apparatus 10 that can operate in a manner to assist patient to acclimatise to the operational level of gas flow (operational) parameters such as humidity and/or temperature. A respiratory apparatus 10 will be described, and then a general example will be described (with examples) in which the respiratory apparatus 10 is configured to implement the humidity and/or temperature acclimatisation. Then several examples will be described. These should not be considered limiting to other variations that could be conceived still provide the described acclimatisation.

The various actions, parameters etc. for any particular example described are not necessarily restricted to just that example and could be used in combination with other examples. The example described are not exhaustive of implementations, and neither are they necessarily independent from each other.

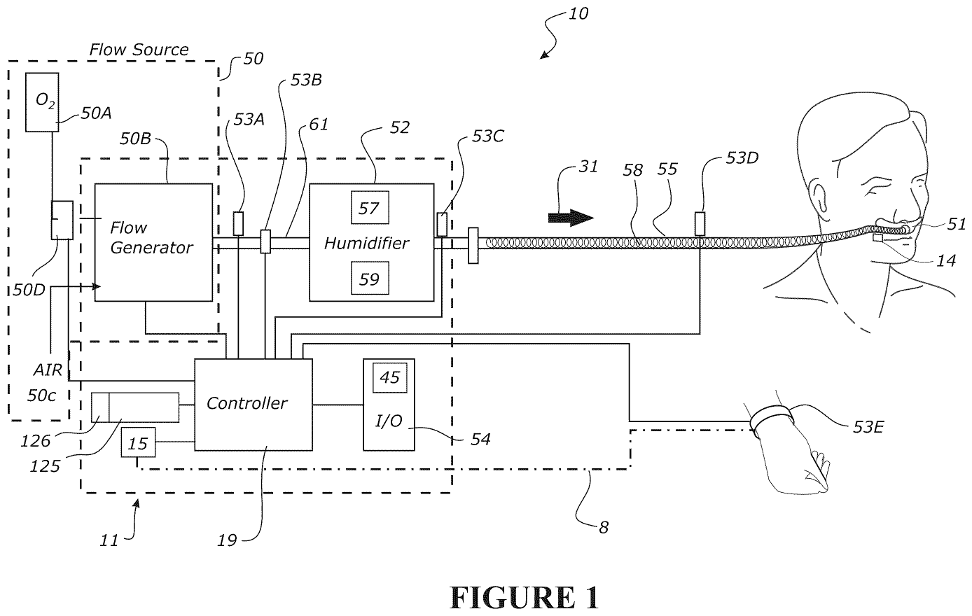

FIG. 1 shows a general example of a respiratory apparatus 10 that uses a conduit 55 with a heater wire 58 and patient interface 51 (sealing or unsealing/non-sealing, depending on use as explained later-a non-sealing is shown in FIG. 1) to provide a gas flow to a patient to provide respiratory support-which can comprise pressure therapy/support and/or optionally flow therapy/support and/or optionally humidification support (see later). The respiratory apparatus is configured for delivering a gas flow 31 at a target pressure and/or flow rate. The breathing gas can be humidified, for example by humidifier 52.

The apparatus has a controller 19 that can be configured to control the operation of the respiratory apparatus 10. The controller 19 can control the apparatus to control the pressure, flow rate, temperature, humidity, oxygen fraction and/or any other operational parameter of gas flow using any suitable control method. To do this, a controller can implement a pressure control method and/or a flow control method to adjust the pressure and/or flow rate and can implement a humidity control method and/or oxygen fraction control method. For example, the controller 19 can control components of the respiratory apparatus 10, including but not limited to: operating a flow generator 50B to create a flow of gas (gases flow) for delivery to a patient, operating a humidifier 52 (if present) to humidify and/or heat the generated gases flow (e.g. by controlling a heater plate of the humidifier), controlling a flow of oxygen into the flow generator blower, operating a heater wire 58 to control temperature and humidity, controlling an oxygen fraction in the gas flow, receiving user input from the user interface 15 for reconfiguration and/or user-defined operation of the respiratory apparatus 10, and outputting information (for example, on the display 54) to the user. It will be appreciated that when the specification describes the apparatus 10 undertaking an action, it may be that the controller 19 is controlling one or more components of the apparatus 10 to undertake the action.

The respiratory apparatus 10 could be an integrated in a single housing such as the dotted box 11, or a separate component based arrangement, generally shown by the individual components within the dotted box 11 in FIG. 1. In some configurations, the apparatus 10 could be a modular arrangement of components 19, 52, 50B. As such, the apparatus could be referred to as a “system” but the terms can be used interchangeably without limitation. Hereinafter it will be referred to as an apparatus, but this should not be considered limiting. If the apparatus 10 is integrated in a single housing, there are advantages of convenience, ease of use, less likely to lose components (especially in a busy hospital environment), improve compliance and the like. It also provides easier humidity and flow control as there is a short flow path between the flow source and humidifier.

Where the apparatus/system 10 is multiple separate components, those components may not be dedicated for use with each other. For example, the disclosure herein could cover use of a standalone humidifier 52, which might be connected to an external flow source 50, e.g. hospital flow source, that itself might be designed for use with different respiratory apparatus. In this case, the humidifier 52 alone might be considered a respiratory apparatus, even before being connected to the flow source, and herein is deemed covered by the term “respiratory apparatus”.

The apparatus 10 may be configured as any suitable apparatus used for any suitable purpose. For example, the respiratory apparatus 10 might be, but not be limited to, a flow therapy apparatus, humidifier respiratory apparatus, a CPAP apparatus, Bilevel/NIV apparatus or the like. The apparatus can be any sort of pressure control and/or flow control respiratory apparatus that provides pressure and/or flow therapy support and/or humidification support. Alternatively, the apparatus may be a multi-therapy apparatus that can provide any combination of one or more of: Nasal High Flow (NHF) therapy, humidifier respiratory therapy, Continuous Positive Airway Pressure (CPAP) therapy and Non-Invasive Ventilation (NIV) e.g. BiLevel pressure therapy. The apparatus 10 may comprise one or more control modes associated with each therapy type. That is, one apparatus 10 can provide any two or more of the therapy types. Alternatively, the apparatus 10 could be dedicated to just one or some of the therapies. The control modes may be manually selected by the user or automatically selected depending on the components connected to the apparatus (for example dependent on the type of tube and/or patient interface connected to the apparatus). Each control mode may have an associated control method for controlling components of the apparatus (for example the flow generator, humidifier heater or conduit heater).

The apparatus components will be described in detail with reference to FIG. 1. FIG. 1 shows various components that can be present for providing flow therapy, CPAP, Bilevel/NIV and/or humidification apparatus that provides one or more of high flow, pressure and/or humidification therapy. Not all components might be necessary for any particular apparatus. An unsealed cannula is shown as an example patient interface 51, but as noted, the apparatus 10 can be configured for one or more other therapies including pressure and/or flow control and/or humidification therapies and a sealed interface might be used instead as appropriate.

The apparatus 10 comprises a flow source 50 for providing a high gas flow 31 such as oxygen, or a mix of oxygen and one or more other gases, providing an oxygen fraction of the gas flow, or more generally a gas fraction. Alternatively, the apparatus 10 can have a connection for coupling to a flow source 50. As such, the flow source 50 might be considered to form part of the apparatus 10 or be separate to it, depending on context, or even part of the flow source 50 forms part of the apparatus 10, and part of the flow source fall outside the apparatus.

The flow source 50 could be an in-wall supply of oxygen, a tank of oxygen 50A, a tank of other gas and/or a high flow therapy apparatus with a blower/flow generator 50B. FIG. 1 shows a flow source 50 with a flow generator 50B, with an optional air inlet 50C and optional connection to an O2 source (such as tank or O2 generator) 50A via a shut off valve and/or regulator and/or other gas flow control 50D, but this is just one option. The description from here can refer to either example. The flow source could be one or a combination of a flow generator 50B, O2 source, air source as described. The flow source 50 is shown as part of the apparatus 10, although in the case of an external oxygen tank or in-wall source, it may be considered a separate component, in which case the apparatus has a connection port to connect to such flow source 50. The flow source 50 provides a (optionally high) flow rate controlled and/or pressure-controlled flow of gas that can be delivered to a patient via a conduit 55, and patient interface 51.

In some configurations, the respiratory apparatus 10 may not comprise a flow generator 50B. In this case the apparatus 10 does not generate a flow of gases, and instead is configured to be connected to an external flow generator and configured to humidify the flow of gases from the external flow generator. For example, the respiratory apparatus 10 can be used as a stand-alone humidifier 52 to humidify gases flowing through the respiratory apparatus 10. The flow generator may be a wall gas supply (regulated via a flowmeter or rotameter, for example) other separate flow generator that can be configured to provide one of the therapies described elsewhere in the specification (e.g., NIV, Bilevel, NHF, CPAP, humidification therapy etc). A user could select one or more of these therapies/modes.

The conduit 55 can have a heater wire 58 that is controllable to heat gas in the conduit 55. The conduit is coupled or couplable at one end to a gases outlet of a water chamber 57 of a humidifier 52 in the housing 11 of the respiratory apparatus 10. Depending on the end-use, the patient interface 51 may be any suitable interface coupled or couplable to the apparatus 10 including one or more of:
an unsealed (also termed “non-sealing”) interface (for example when used in high flow therapy) such as a nasal interface (cannula), with a manifold and nasal prongs, and/or a face mask, and/or a nasal pillows mask, and/or a nasal mask, and/or endotracheal tube, and/or a tracheostomy interface, or any other suitable type of patient interface; or
a sealed interface (for example when used in NIV, CPAP) such as a nasal mask, full face mask, or nasal pillows.

A humidifier 52 with a water chamber 57 and base/heater plate 59 can be provided between the flow source 50 and the patient to provide humidification of the delivered gas. This could be a humidifier integrated with the flow source 10 to form an integrated apparatus 11 (see dotted lines) or separate but attachable/detachable to/from the flow source 10 (e.g. flow generator). The heater plate may be or comprise a heater element, or heating surface, or other apparatus suitable for imparting heat to a liquid, such as water. The heater plate may have an exposed heating surface, be formed from a plurality of layers including at least one heating element, and/or form art of the base of the humidifier. In each case the heater plate is configured to evaporate water, whether from a water chamber directly, from a dosed amount of water placed on the heater plate, or otherwise.

Alternatively, the humidifier 52 could be a standalone humidifier (connectable/disconnectable to/from the flow source) with a water chamber 57 and base, where the humidifier is coupled to the flow source 10 via conduits 61 or other suitable means. The apparatus 10 (or the humidifier 52 when standalone) may comprise one or more sensors and a controller that is configured to control the humidifier based on the sensor measurements. The humidifier controller may be the same as, combined with, part of, or linked to the apparatus controller 19. For example, the humidifier 12 may comprise a temperature sensor and a flow sensor. The humidifier 12 can humidify the gases flow and/or heat the gases flow to an appropriate humidity/temperature level. The controller (for example apparatus controller 19) can be configured to control the humidifier 52 (for example, by controlling at least a humidifier heater). The humidifier 52 may be optional, or it may be preferred due to the advantages of humidified gases helping to maintain the condition of the airways. Humidification is optionally used with high flow gas flows to increase patient comfort, compliance, support and and/or safety.

The heater wire 58 can be controlled by the controller to heat the gas flow 31 to further control the temperature and/or humidity of the gas flow.

One or more sensors 53A, 53B, 53C, 53D, 53E, 14 such as flow rate, oxygen fraction, pressure, humidity (dew point, RH or AH), temperature or other sensors can be placed throughout the apparatus and/or at, on or near the patient. Alternatively, or additionally, sensors from which such parameters can be derived could be used. In addition, or alternatively, the sensors 14, 53E can include one or more physiological sensors for sensing patient physiological parameters such as, heart rate, oxygen saturation (e.g. pulse oximeter sensor/SpO2 53E), partial pressure of oxygen in the blood, respiratory rate, partial pressure of O2 and/or CO2 in the blood. Alternatively, or additionally, sensors from which such parameters can be derived could be used. Other patient sensors could comprise EEG sensors, torso bands to detect breathing, and any other suitable sensors. One or more of the sensors might form part of the apparatus, or be external thereto, with the apparatus having inputs for any external sensors. The sensors could be in any suitable place for what they are sensing, comprising, but not limited to: flow generator or source inlet or outlet, humidifier inlet or outlet, heater plate 59, conduit 55, patient end of conduit (end of hose “EOH”), patient interface 51 and/or patient. FIG. 1 shows a sensor 53A at the output of the flow generator, and a sensor 53B between the flow generator and the humidifier. Sensors 53A and 53B are shown on conduit 61. Sensor 53B may be arranged at the inlet to the humidifier 52. Sensor 53C is arranged at the outlet of the humidifier, or at the inlet of the conduit 55. Sensors 53D is arranged on the conduit 55. In some cases it may be at or towards the patient interface 51 end of the conduit. Sensor 53E is configured to attach to the patient. Further sensors may be used where helpful, for example to provide good feedback to controller 19 or sense a parameter of the gases flow. In some cases an ambient sensor is located before the flow generator 50B.

The output from the sensors is sent to a controller 19 to assist control of the apparatus 10, comprising, among other things, to control:
the flow generator 50B, or flow source 50, to control flow rate and/or pressure of the gas flow
the humidifier and/or heater wire to control temperature and/or humidity of the gas flow
the oxygen fraction of the gas flow alternatively, or additionally, input could come from a user.

The controlled parameter such as humidity, temperature, flow, pressure, oxygen fraction or the like can be controlled at any suitable point, such as at the end of the breathing conduit 55, at the patient interface 51, at the gases outlet, a humidification chamber outlet, at any sensor 53A to 53D of the apparatus, any other place where the sensor(s) might be as noted above and/or any combination thereof. The parameters can be controlled to a set point (target). These set points could comprise (just a small subset of examples without limitation), a desired dew point (for example a temperature indicative of a desired humidity), a predetermined dew point, a predetermined temperature, a desired temperature.

The controller 19 may be coupled to the flow source 50, humidifier 52 and sensors 53A to 53D and/or any other component to achieve control. It controls these and other aspects of the apparatus to be described below.

The controller 19 can also control any other suitable parameters of the flow source 50 to meet oxygenation requirements/fraction—such as mixing valves such as a proportional valve. The controller 19 can also control the humidifier 52 based on feed-back from the sensors 53A-53E, 14. Six are shown by example but any suitable number could be used and placed anywhere suitable. Using input from the sensors, the controller 19 can determine oxygenation requirements and provide information to a medical professional (who may control the components of the respiratory apparatus 10 to provide the desired therapy, e.g. flow rate, O2 fraction, humidity, etc.) and/or control parameters of the flow source, temperature and/or humidifier as required.

The controller 19 is also configured to operate the apparatus 10 so that the flow, pressure, volume and/or other parameters of gas provided by the flow source based on feedback from sensors, or optionally without feedback (e.g. using default settings). The controller 19 can also control any other suitable parameters of the flow source 50 to meet oxygenation requirements. The controller 19 may use a sensor feedback loop. At a high level this includes collecting new sensor measurements, using the sensed metrics to determine the current state of the gas in the device, comparing the current state to the target values, and adjusting the system outputs to achieve the target value.

The controller 19 could take input from many sensors as feedback in the ramp progress (e.g., a humidity ramp process). These inputs could include: humidity, ambient temperature, pressure, temperature of the air at the humidifier inlet, temperature of the gas at the humidifier outlet, temperature of the heater plate, power supplied to the heater plate, flow rate and inlet dew point.

An input/output interface 54 (such as a display and/or input device) is provided (“user interface”). The input device is for receiving information from a user (e.g., clinician or patient) that can be used for example for determining oxygenation requirements, anaesthetic gas agent, detection, flow rates, gas fractions, partial pressures and/or any other parameter that might be controlled by the apparatus 10.

The apparatus 10 also comprises a display 45 which can be part of the I/O 54 for displaying the measure of the gas parameter of the exhaled gas flow, as a graph, digital readout or any other suitable means. It could display any sensor information and/or operational parameters, such as for example humidity, temperature, flow rate, pressure, oxygen fraction, SpO2 or the like.

The respiratory apparatus 10 may have a communications module 15 to enable the controller 19 to receive signals 8 from the sensors and/or to control the various components of the breathing assistance apparatus 10, including but not limited to the flow generator 50B, humidifier 52, heater, humidifier heater 59, or accessories or peripherals associated with the respiratory apparatus 10. Additionally, or alternatively, the communications module 15 may deliver data to a remote server or enable remote control of the respiratory apparatus 10 or respiratory therapy system. The communications module 15, which may be referred to as a transceiver, can transmit various information e.g. usage information, amount of times comfort mode was initiated, parameters of comfort mode (e.g. time period for ramp, being a ramp rate) and operational mode parameters e.g. humidity, temperature, flow rate, etc.

The communications module 15 may comprise a transmitter, receiver and/or transceiver. It could e.g. be a modem, WIFI™ transceiver, BLUETOOTH™ transceiver or any other suitable transceiver.

The communications module 15 may act as a network interface (for example, as a modem).

The respiratory apparatus 10 may comprise or be in the form of a high flow therapy apparatus. When configured to provide high flow therapy, the one or more operational parameters for high flow therapy (also termed nasal high flow therapy or NHF therapy) may comprise any combination of:
a therapy flow rate of the gases provided to the user,
a therapy humidity level (for example a relative or absolute humidity, or a dew point)
a therapy concentration of an auxiliary gas (e.g. O2) provided to the user,
a therapy temperature of the gases provided to the user (for example).

High flow therapy, as discussed herein, is intended to be given its typical ordinary meaning as understood by a person of skill in the art which generally refers to a respiratory apparatus delivering a targeted flow of humidified respiratory gases via an intentionally unsealed patient interface with flow rates generally intended to meet or exceed inspiratory flow of a patient. Typical patient interfaces include, but are not limited to, a nasal or tracheal patient interface. Typical flow rates for adults often range from, but are not limited to, about fifteen litres per minute to about sixty litres per minute, or greater. Typical flow rates for paediatric patients (such as neonates, infants and children) often range from, but are not limited to, about one litre per minute per kilogram of patient weight to about three litres per minute per kilogram of patient weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other common names.

For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient, ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may, in some configurations, deliver gases to the patient at a flow rate of between about 1 LPM and about 100 LPM, or at a flow rate in any of the sub-ranges outlined above. Gases delivered may comprise a percentage of oxygen. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%.

High flow therapy may be effective in meeting or exceeding the patient's inspiratory flow, increasing oxygenation of the patient, and/or reducing the work of breathing. High flow therapy may be administered to the nares of a patient and/or orally, or via a tracheostomy interface.

High flow therapy may generate a flushing effect in the nasopharynx such that the anatomical dead space of the upper airways is flushed by the high incoming gases flow. This can create a reservoir of fresh gas available for each and every breath, while reducing re-breathing of nitrogen and carbon dioxide. Meeting inspiratory demand and flushing the airways is additionally important when trying to control the patient's FdO2 (Fraction Delivered in Oxygen). High flow therapy can be delivered with a non-sealing patient interface such as, for example, a nasal cannula. High flow therapy may slow down respiratory rate of the patient. High flow therapy may provide expiratory resistance to a patient.

High flow therapy may be used to treat patients with obstructive pulmonary conditions e.g., COPD, bronchiectasis, dyspnea, cystic fibrosis, emphysema and/or patients with respiratory distress or hypercapnic patients.

The respiratory apparatus 10 may comprise or be in the form of a Bilevel/NIV respiratory apparatus. Bilevel therapy may comprise providing gases to a user at a therapy IPAP and EPAP (and optionally one or more operational parameters as described in more detail below.). The respiratory apparatus 10 may comprise or be in the form of a CPAP respiratory apparatus that provides CPAP therapy. CPAP therapy may comprise providing gases to a user at a constant pressure. The respiratory apparatus 10 may comprise or be in the form of a humidification respiratory apparatus that provides humidification therapy.

In some configurations, the apparatus may comprise at least one battery as part of a battery module 125 (with optional battery cover 126). The battery module 125 may be located in the housing 11 of the apparatus 10, and/or attached externally to the housing 11 of the apparatus. It will be appreciated when the term battery is used in the specification it may refer to either the battery itself, or the battery module 125 which comprises the battery. The battery module 125 can supply power when mains is unavailable (as a battery supply). For example, this might allow the patient to move around with the apparatus 10 and still get therapy and/or the comfort mode. In some configurations, the battery may be removably coupled to the apparatus and is rechargeable. In some configurations, the battery is removable and optionally connectable and disconnectable from the apparatus 10. Alternatively, the battery is non-removable. In some configurations, the battery is provided as part of the same housing as the flow generator and/or the humidifier. In some configurations, the battery is provided as connectable and disconnectable to the same housing as the flow generator and/or the humidifier. Having the battery as part of the housing, or connectable and disconnectable to the housing may allow for the apparatus to be portable compared to other apparatuses (for example larger apparatuses such as ventilators, or those with external battery power sources which are not portable). Portability of the apparatus may increase usability of the apparatus in a homecare setting as the apparatus can be more easily moved around the user's house. In a hospital setting portability allows the therapy apparatus to be moved around the hospital with the patient so the patient can continue to receive therapy while being transported.

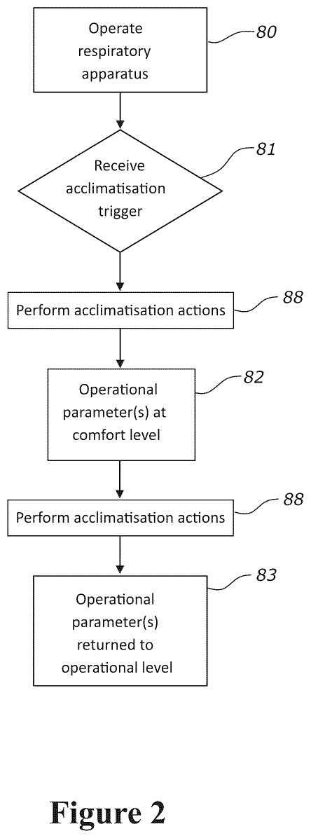

The respiratory apparatus 10 of FIG. 1 in any of its forms can be configured to provide humidity and/or temperature gas flow parameter (“humidity and/or temperature”) acclimatisation as shown in the method of FIG. 2. The controller 19 of the apparatus 10 to operate the apparatus as described. Typically gas flow (operational) parameter acclimatisation will related to gas flow temperature and/or humidity acclimatisation (and the description will focus on these parameters by way of example).

The respiratory apparatus 10 is configured to, at the appropriate time, acclimatise the patient to operational levels of humidity and/or temperature (operational parameters) by controlling the temperature and/or humidity of the gas flow over time until so the patient feels comfort, and therefore is more likely to be compliant. The time period is long enough/the ramp is slow enough to allow the patient to get used to the humidity and/or temperature.

The description refers to humidity and temperature. It should be reiterated that humidity and temperature are operational parameters, and reducing or increasing humidity and temperature means reducing or increasing an operational parameter level. An operational parameter can take many levels (values), including an initial comfort level, an operational level or any level in between. The operational level is the operational parameter level (value e.g. degrees or dew point or percentage relative humidity) provided during usual operation/therapy. For example, operational level parameter value could be 37 degrees C dewpoint at 100% RH. Other dewpoint settings could be 31 degrees Celsius, 34 degrees Celsius.

In general terms, the respiratory apparatus 10 is configured to activate an acclimatisation method which implements acclimatisation actions to provide acclimatisation outcomes. That is, it alters the temperature and/or humidity of gas over time (method) from an initial comfort level to an operational level to provide comfort to/acclimatise the patient (acclimatisation outcomes). This initial comfort level of the humidity and/or temperature is comfortable/tolerable to the patient. As the parameters rise to the operational level, the patient can acclimatise so that the gas flow 31 is comfortable when they reach the operational level. Activating the acclimatisation method can be deemed as putting the apparatus into a “comfort mode” of operation. Some examples of initial comfort level parameter values could be (typical non-limiting example): a certain number of degrees below operational level dew point. For example, the initial comfort level dew point could be 3 degrees below operational dew point level, e.g. comfort level is 34 degrees dew point when the operational level is 37 degrees dew point. Any suitable drop from operating level dew point is possible.

The respiratory apparatus 10 may do this by implementing acclimatisation actions. Acclimatisation actions may control the heater plate 59 and/or heater wire 58 to control gas flow 31 humidity and/or temperature. In general terms, an acclimatisation action might comprise controlling any one or more of:
the heater plate 59 so that the temperature and/or humidity of the gas flow 31 reduces to an initial comfort level then increases over time to an operational level, and
the wire 58 so that the temperature and/or humidity of the gas flow 31 reduces to an initial comfort level then increases over time to an operational level, and
both the heater plate and the wire, and
the flow generator 50B and/or other components of the humidifier 52 or respiratory apparatus 10.

Controlling the heater plate 59 and/or heater wire 58 may comprise controlling power to the heater plate 59 and/or heater wire 58 as required. This can be done in various ways. This could be by way of controlling voltage and/or current to the heater plate 59/heater wire 58, such as by controlling duty cycle, voltage/current magnitude and/or any other suitable method.

The acclimatisation actions may be implemented in various ways, with some non-limiting examples being:
modifying closed loop control of humidity and/or temperature at the water chamber outlet,
modifying set point temperature and/or humidity at the chamber outlet and/or patient end,
open loop control of power provided to the heater plate and/or heater wire, and
closed or open loop of some other parameter that indirectly controls temperature and/or humidity at the water chamber outlet and/or patient end of the conduit 55.

Referring to FIG. 2, a general acclimatisation method will be described. At a suitable time during operation, step 80, of the respiratory apparatus 10, the respiratory apparatus can be triggered, step 81, to provide temperature and/or humidity acclimatisation (“acclimatisation” or “acclimatisation method”). The respiratory apparatus 10 then performs acclimatisation actions, step 88, to alter the operational parameters to a comfort level, step 82, before, potentially after a delay or other action, initiating one or more acclimatisation actions, step 88, to return the operational parameter(s) to the operational level, step 83.

The trigger of step 81 could be a mode of operation and could be called an “acclimatisation mode”, “comfort mode” or some other label indicating the function/purpose of the mode. As noted earlier, this is configured to encourage patient comfort and acclimatisation to the therapy, and therefor to provide better compliance. Acclimatisation could happen or be triggered to happen at any time, for example:
immediately on turning on the machine (“start-up”);
triggered by a patient;
triggered by a patient to occur during a warmup period/mode (when the respiratory apparatus gets up to temperature). The warm-up mode could be triggered upon start up and/or from a standby mode. The warm-up mode could be triggered at some other point. The warm-up mode may be a non-operational level period; or
during the operational period (including at the start of the operational period).

Typically, the respiratory apparatus 10 will be triggered to provide acclimatisation at a stage after the warmup period, such as during the operational period, although that is not essential. The apparatus 10 can be triggered, step 81, into the acclimatisation method manually (e.g. by patient or other operator interacting with the respiratory apparatus through the user interface 54, communication interface 15, or similar) or automatically (e.g. by the respiratory apparatus detecting some current operational state that suggests acclimatisation operation should occur). These options will be described in detail later.

As part of the acclimatisation method, humidity and/or temperature (operational parameters) will be varied (using acclimatisation actions 88). The variations may be configured to create variation of the humidity and/or temperature of the gas flow. Acclimatisation actions 88 are operations of the respiratory apparatus 10 used to vary the humidity and/or temperature to implement the acclimatisation method and achieve acclimatisation outcomes.

In some cases, to determine:
whether acclimatisation outcomes are required (e.g. if humidity and/or temperature need varying to achieve acclimatisation), and/or
what acclimatisation actions to take,
   one or more of the following characteristics may be taken into account. The heater plate 59 is the largest contributor to the humidity of the gas flow 31. Typically, in humidity control of a gas flow 31, the heater plate 59 temperature determines the rate of evaporation of the water in the humidification chamber 52, and thus is a driver of humidity in the gas flow 31. It also contributes to the gas flow temperature. The heater wire 58 controls gas flow 31 temperature in conduit 55 (which may be referred to as a breathing tube) to keep the gas flow at a temperature a few degrees above the estimated dew point to prevent condensation forming. Generally, the conduit 55 temperature is the main contributor to the temperature of the gas to the patient (i.e., at or near the patient interface 51).

The acclimatisation actions 88 are implemented by the controller 19 controlling the respiratory apparatus 10 to alter the humidity and/or temperature parameter(s) as required to achieve acclimatisation outcomes. Acclimatisation outcomes may include varying of a humidity and/or temperature parameter, such as increasing/decreasing humidity and/or temperature. For example, an acclimatisation action 88 could be the heater plate 59 is controlled to control humidity and/or temperature from an initial comfort level then raise it to an operational level. Alternatively, or in addition, the heater wire 58 may be controlled to control humidity and/or temperature from an initial comfort level then raise it to an operational level (including any parameter maintenance period).

Some example acclimatisation actions 88 for a typical acclimatisation method will now be discussed. It will be understood that these are not limiting. Once the respiratory apparatus 10 is triggered to implement acclimatisation (step 81), insofar that the humidity and/or temperature parameters of the respiratory apparatus 1 are above/not at an initial comfort level, one or more of the humidity and/or temperature parameters are decreased to an initial comfort level, step 82, (the initial comfort level being where the operational parameter is at a level that the user can tolerate). If the respiratory apparatus 10 is already operating or otherwise providing operating parameters at the operational initial comfort level, then this decreasing step is not required). In other cases, the comfort level may be configured based on the operating level. For example, a predefined level below the operating level, or a percentage reduction from the operating level.

The humidity and/or temperature parameters are decreased to the initial comfort level by operating the heater plate 59 and/or heated conduit 55 (acclimatisation action) to reduce the gas flow temperature and/or gas flow humidity (acclimatisation outcome). For example, to decrease the humidity and/or temperature parameters to the initial comfort level one or more of the following example acclimatisation actions can be implemented to achieve acclimatisation outcomes:
the heater plate power is reduced to reduce gas flow humidity at the water outlet and/or the patient interface;
the heater plate power is reduced to reduce gas flow temperature at the water chamber outlet and/or the patient interface;
the heater plate power and/or heater wire power is reduced to reduce the gas flow temperature at the patient;
The humidity is reduced but as a result temperature rises, so the temperature is reduced by reducing the heater plate and/or heater wire power;
The heater wire is controlled to change the relative humidity;
The heater plate and heater wire can be controlled to reduce dew point.

It should be noted that when changing the humidity level by reducing/increasing humidity, that refers to a humidity parameter such as dew point, absolute humidity or relative humidity or any other measure being or indicative of some type of humidity. The exact nature of the parameter used is not critical to invention.

The humidity and/or temperature might optionally be maintained at the comfort level for a maintenance period. Over time, the opposite action (or one or more acclimatisation actions 88 to achieve the opposite outcome of the initial acclimatisation action 88) is taken to increase (“ramp”) the humidity and/or temperature of the gas flow back to the operational level 83. (Reference to increasing a humidity and/or temperature parameter over a time period can be referred to as a “ramp” or “ramp rate”.) The time period may be configured long enough/the ramp is slow enough to allow the patient to get used to the humidity and/or temperature at each stage or step of the ramp, or to not notice the slow increase.

Operational parameters may be increased by operating the heater plate 59 and/or heated conduit 55 to increase the gas flow temperature and/or gas flow humidity. For example, to increase the humidity and/or temperature from the initial comfort level to the operational level, one or more of the following acclimatisation actions can be implemented to achieve acclimatisation outcomes:
the power provided to the heater plate is increased over a time period to increase gas flow humidity at the water outlet and/or the patient interface to operational level;
the power provided to the heater plate is increased over a time period to increase gas flow temperature at the water chamber outlet and/or the patient interface to operational level;
the power provided to the heater plate power and/or heater wire is increased over a time period to reduce the gas flow temperature at the patient;
The heater wire is controlled to change the relative humidity;
The heater plate and heater wire can be controlled to reduce dew point.

It should be noted that the list of acclimatisation actions 88 above for controlling heater plate and heater wire down to the initial comfort level and back to the operational level is not exhaustive of the acclimatisation actions 88 that could be used to achieve acclimatisation outcomes. Further, two or more of the acclimatisation actions 88 could be used in combination to effect acclimatisation. In some cases different acclimatisation actions 88 are used to decrease and increase the same operational parameters, even where the same operational level is returned to, step 83.

Some aspects of the acclimatisation method will now be described in more detail. In one manual (user input) trigger option of step 81, the patient (or other operator) might determine that they are experiencing discomfort, so wish to activate the acclimatisation method. For example, the respiratory apparatus 10 might be in normal operation mode, providing operational level parameters, and the patient may consider that the gas flow is uncomfortable due to one or more of the humidity and temperature being too high. The patient could trigger the acclimatisation method 81 by operating the user interface 45. For example, the respiratory apparatus 10 could have a “comfort” button or similar (e.g. a push button, a sequence of button presses, or access a menu, or a rotatable dial that can be used or the like) that is provided on the user interface. Pressing the comfort button will instruct the controller 19 to operate the acclimatisation method.

In another automatic (internal) trigger option, step 81, the respiratory apparatus 10 itself might determine that the acclimatisation method is required, based on some operational condition that suggests that the patient is experience discomfort with the operational parameters at the current operational level. In doing so, it triggers acclimatisation actions 88 to the comfort level, step 82.

In one option, the controller 19 detects if a patient interface 51 has been removed from patient's face a plurality of times (e.g. 3 times) in the same therapy session or in a rolling timeframe. Removal may be detected by a change in air flow, a sensor, or otherwise. Removal of the patient interface 51 multiple times in the same session is indicative of discomfort of the therapy for a patient. The frequency and duration of each interface 51 removal could be tracked. If multiple removals are detected in a single therapy session, then a comfort button or similar is provided on the user interface 45 to allow a patient to active the acclimatisation. Alternatively, if multiple removals are detected in a single therapy session the acclimatisation can be automatically activated. Either could be started or suggested to the patient once a threshold of removals reached, or number of removals in a rolling timeframe, or the time duration of a removal. In a further example, the acclimatization could be automatically activated on any removal detection, or when the patient interface 51 is detected as being replaced on a patient's face.

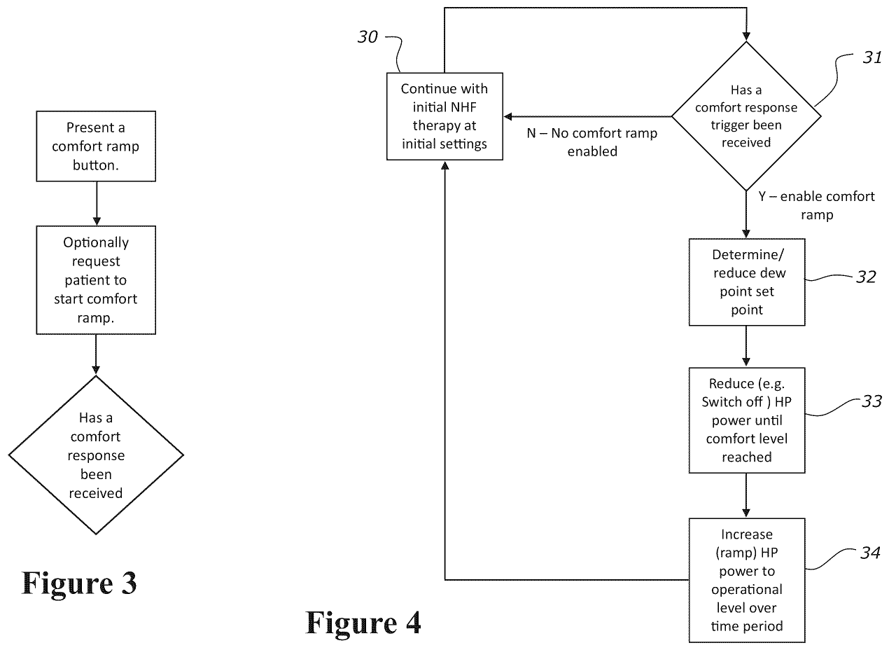

In another option, the controller 19 detects if a patient interface 51 has been removed permanently. Removal of the patient interface 51 indicates the patient is in discomfort and has stopped using the apparatus/receiving therapy. When a patient receiving therapy is uncomfortable, a natural response is to remove the interface providing the gas. The respiratory apparatus 10 can determine whether a patient is using an interface, and when they are not, for example by a sensor or change in flow. The frequency and duration of the removal could be tracked. If removal is detected, then referring to FIG. 3 a comfort button or similar is provided on the user interface to allow a patient to active the acclimatisation. An alert, such as an audio alert, or visual alert could encourage the comfort button to be pushed. Alternatively, if a removal is detected, the acclimatisation can be automatically activated.

A patient interface 51 removal, such as a nasal cannula removal, can be detected based on any one or more of the following:
Sensing flow fluctuations at a flow sensor (e.g. internal flow sensor) in the respiratory apparatus. The flow fluctuations are sensed and compared with a reference threshold or a reference waveform;
Analyzing gas flow signals to determine a respiratory rate of the patient—an absence of breathing patterns in the data could suggest the mask is not being worn;
Using a pulse oximeter or other physiological sensor embedded in the breathing apparatus—for example, the absence of a signal could be indicative that the patient is no longer wearing the interface;
The pressure fluctuations of the gas in the conduit could be used to detect the breathing cycle of a patient wearing the interface. For example, this could be measured directly with a pressure sensor or by examining the motor speed oscillations.

Once the threshold or criteria for patient interface 51 removal has been met, an alert could be initiated to indicate to the patient that the acclimatisation method could be used or will be used.

As described above, when acclimatisation is triggered, step 81, the gas flow operational parameters such as humidity and/or temperature may be altered as part of acclimatisation, using one or more acclimatisation actions 88 to achieve one or more acclimatisation outcomes. For example, as part of acclimatisation, the humidity and/or temperature parameters might be reduced (action) so that the humidity and/or temperature parameters are at an initial comfort level (outcome), step 82. After a comfort level has been achieved step 82, the parameters might be increased (acclimatisation actions 88) to be at an operational level (outcome), step 83. In some cases acclimatization actions may increase and decrease during the ramp, so as to soften the approach to the operational level or encourage patient comfort. The acclimatisation actions 88 themselves can be implemented in various ways, by control of components of the respiratory apparatus 10.

The discomfort is often caused by humidity; however, it could be caused by any combination of the operational parameters (e.g. humidity, temperature). The acclimatisation action can be implemented in any suitable manner to address the discomfort. Typically, the humidity is controlled by controlling the heater plate 59 to a particular dew point/humidity at the outlet of the water chamber 57; and/or typically the heater wire 58 is controlled to control the temperature at the patient end. Often these are used in combination. However, these are not essential control methods, and any suitable control of heater plate 59 and/or heater wire 58 could be used to control humidity and/or temperature to implement the acclimatisation method/acclimatisation actions.

For example, the heater plate 58 could be used to control temperature and/or the heater wire 59 could be used to control humidity. Also, control of temperature might be used to control humidity and/or control of humidity can be used to control temperature. Some general, but non-limiting, examples of acclimatisation actions 88 to achieve steps 82 and 83 comprise:
controlling the humidity by one or more of:
   controlling the humidity set point (dew point, relative humidity or absolute humidity) at the outlet of the water chamber and using closed loop control provide power to the heater plate to achieve the changing humidity set point;
   controlling the temperature set point of the heater plate and using closed loop control provide power to the heater plate to achieve the changing heater plate temperature set point; and
   ramping power to the heater plate (e.g. using changing duty cycle) to control the humidity using open loop control; and/or
Controlling the temperature by one or more of:
   controlling the temperature set point at the patient end and using closed loop control provide power to the heater wire to achieve the changing humidity set point;
   controlling the temperature set point of the heater wire and using closed loop control provide power to the heater wire to achieve the changing heater wire temperature set point; and
   ramping power to the heater wire (e.g. using changing duty cycle) to control the temperature using open loop control.

Some exemplary example of acclimatisation actions, 88, for explanatory purposes, are now provided.

Where closed loop control is used to by the respiratory apparatus 10 to control temperature and humidity to a set point (that is target temperature or humidity), the acclimatisation action 88 can be revising the set point. The acclimatisation actions 88 can also be the ensuing closed loop control that controls to the new set point. Of course, where gradual increasing of a humidity and/or temperature parameter is part of the acclimatisation action 88, that might comprise gradually increasing the set point.

As an example, the gas flow humidity might be reduced to an initial comfort level 82, and then increased to an operational level 83 over a time period. That is, the humidity can be ramped to the operational level. To achieve this, the power to the heater plate is ramped to ramp the heater plate temperature (which ramps humidity of the gas flow). To do this, the heater plate power is ramped over a period of time. The controller 19 controls the heater plate power to ramp according to a profile over the time period. The time period might be specified as described later. The time period will define the rate of change of the humidity ramp. A longer time period allows for a slower humidity ramp and more time for the patient to acclimatise.

There are many ways to control the heater plate 59 and/or heater wire 58, typically by controlling power to the heater plate/heater wire, via voltage and/or current control. In one typical, but not limiting, operation of a respiratory apparatus 10, the heater plate 59 is controlled by a PID controller (Proportional-Integral-Derivative Controller), wherein the increase from ambient temperature to the temperature needed to achieve the required dewpoint is as fast and accurate as possible. Implementing a humidity ramp using a PID control could be achieved in a few ways.

In a first method, when the acclimatisation method is initiated 81 and the initial comfort level has been reached 82, a PID controller with different parameters is used to ramp the humidity back up to the operational level. If the PID settings used in normal device operation are tuned to resemble ideal control (minimal rise-time, minimal overshoot, no steady-state error), then PID settings applied when the humidity is ramping would be tuned differently so the rise time is a longer period (that could be set by the patient). This could be achieved by reducing the proportional and integral gain constants.

Another method to control the acclimatisation is to limit the voltage or current supplied to the heater plate to reach the initial comfort level. The controller 19 determining the operation of the heater plate 59 determines the power to be supplied to achieve the desired temperature (operational level of temperature) and consequent dew point (operational level of humidity) of the gas. The rate of temperature increase can be slowed by scaling the outputs of the PID controller or capping the maximum power so that the system retains the favorable steady state conditions present with optimal PID tuning while having a longer time to reach the target temperature.

Another method of control is to measure temperature at the water chamber outlet (e.g. with a sensor 53C, which may be a temperature sensor) of the humidification chamber 52 (or measure at an elbow in the conduit 55 where it couples to the respiratory apparatus 10) and control to a changing target temperature (set point from an initial comfort level to an operational level). The target temperature is set by a user (e.g. a clinician) and the controller 19 controls the heater plate 59 using closed loop control such that the gases temperature is at the target temperature when measured at the chamber outlet. The target temperature is assumed to be dew point so that this acclimatisation action 88 achieves a humidity control from an initial comfort level 82 to an operational level 83.

In another method, the changing target temperature is set by the user. A target flow rate is set by the user. The controller 19 calculates a target heater plate temperature and controls to the target heater plate temperature by measuring the heater plate temperature from a heater plate temp sensor.

In another method the voltage or current may be ramped upward or the voltage or current set point may be ramped up slowly. In a further alternative the duty cycle applied will be ramped at a set rate. The heater plate 59 is powered by duty cycle control. The duty cycle may be reduced to achieve the lower temperature and then the duty cycle target is ramped to achieve the humidity/temp ramp.

Alternatively, the difference between the target dew point and the lower starting point at the beginning of the humidity ramp could be fed into the control system as many incremental increases in the target dew point, rather than a single change. The size of the increments and the delay between consecutive increases would allow the total ramping period to be predictably set. Using this method, the existing optimal PID tuning can be used at each increase in set point up to the desired dew point.

In another method, the respiratory apparatus 10 is operated to provide operational parameters at prescribed level, the prescribed gas flow temperature, humidity and flow rate. The patient might find the temperature and/or humidity at the patient end is uncomfortable, and therefore operates the apparatus to trigger the acclimatisation method. One of the acclimatisation outcomes is to ramp humidity of the gas flow to gradually acclimatise a patient to their prescribed therapy settings. It does this by controlling the heater plate 59 (acclimatisation action 88) through slow ‘ramping’ of humidity settings from a lower setpoint up to the operational settings (acclimatisation action). In some examples the ramping function could be initiated when the patient is experiencing discomfort. This causes the controller 19 to reduce power to the heater plate 59, so the heater plate temperature drops, and the humidity of the gas flow drops to a comfort level humidity. The controller 19 gradually over a period of time brings up (ramps) the power to the plate under closed loop control as the lower set point goes from a lower level to an operational set point, so that they temperature slowly increases, thus increasing the humidity of the gas flow until it reaches an operational level. Alternatively, the controller 19 might reduce power to the heater wire so as to reduce the temperature of the gas flow, and then gradually over the period of time increases power to the wire to increase low-temperature.

For example, the set point might not be gradually increased, but rather set at the operational level, and then another control method used to increase power to the heater plate 59 and/or heater wire 58 in order to increase the humidity and/or temperature parameters to the operational level (e.g. by changing the duty cycle).

In another method, the heater wire 58 is controlled to ensure the temperature stays above the dew point. Generally set point at patient end is a pre-set amount above the chamber outlet set point. E.g. 3 degrees C more. This way you reduce condensation. The heater wire can be controlled to change the humidity or the temperature of the gas flow.

Of course, these are non-limiting examples. There are other control methods known to those skilled in the art, and acclimatisation as herein described can be implemented using any suitable control method that implements the acclimatisation method/actions that can appropriately control humidity and/or temperature parameters to achieve acclimatisation for the patient.

When implementing acclimatisation actions 88, the controller 19 can use one or more sensor 53A-53E, 14 inputs to obtain the required information. The controller 19 uses the same sensor feedback loop as in normal operating modes. At a high level this includes collecting new sensor measurements, using the sensed metrics to determine the current state of the gas in the device, comparing the current state to the target values, and adjusting the system outputs to achieve the target value.

The controller could take input from many sensors as feedback in the humidity ramp progress. These could include any one or more of:
Humidity;
Ambient Temperature;
Pressure;
Temperature of the air at the humidifier inlet;
Temperature of the gas at the humidifier outlet;
Temperature of the heater plate;
Power supplied to the heater plate;
Flow rate; and
Inlet dew point.

These sensors could take readings at any suitable point, such as any one or more of:
the humidifier inlet;
in the humidifier;
the humidifier outlet;
the breathing conduit inlet;
in the breathing conduit;
the breathing conduit outlet;
a patient interface inlet;
in the patient interface; and
a patient interface outlet.

Acclimatisation has a time period. This is the time over which the acclimatisation actions 88 are implemented. The time period can influence the time profile of the varying of a humidity and/or temperature parameter. The acclimatisation time period can be determined in a suitable manner to achieve acclimatisation of the patient to the operational level of humidity and/or temperature parameters. Optionally the time period comprises a maintenance period where the apparatus maintains the initial comfort level, before ramping to the operational level. The maintenance period could instead be additional to the ramp time period.

For example, the user (e.g. patient or clinician) may set the time period manually (or multiple time periods), or there might be a plurality of time periods that a patient can select from. For example, there could be a short, medium and long time period. These time period settings may be predefined and stored in the memory of the flow generator.

Note, there can be a different time period for varying temperature and for varying humidity or varying other humidity and/or temperature parameters. In such cases, there is an overall acclimatisation time period over which all humidity and/or temperature parameters are varied to achieve acclimatisation, but the actual time period over which one of them is varied might be a separate, shorter time period.

The time period(s) can be defined based on various factors that will assist patient acclimatisation. Factors can comprise:
How quick the response of machine is, and
The time it takes patient to build tolerance to humidity and temperature.

Some time periods can include:
at least about 10 minutes,
at least about 15 mins,
at least about 30 minutes,
between about 30 minutes and about 40 minutes,
about 35 minutes,
at least about 40 minutes
about 45 minutes
between about 30 minutes and about 50 minutes,
between about 30 minutes and about 60 minutes
between about 35 minutes to about 1 hour,
at least about 1 hour,
between about 1 hour and about 2 hours, or
up to about 2 hours
calculated from a rate of about 1 degree C/5 minutes, to ramp from initial comfort level to the operational level (e.g. 31 degrees dew point to 37 degree C dewpoint would take about 30 minutes) In some cases the acclimatisation mode might not immediately ramp. Rather the apparatus may provide/maintain the initial comfort level for some time (which may be referred to as a maintenance period), before ramping to the operational level. For example, it could be maintained at the comfort level for at least about 10 minutes or at least about 15 minutes or for about 30 to 60 minutes before the ramp as per the time period above. The time period can be deemed to cover the maintenance period at which the initial comfort level is maintained, or the maintenance period might be additional to the time period.

The preferences of the patient/user may be stored in the memory of the respiratory apparatus 10 as part of a patient profile. The patient profile may alternatively or additionally be stored at a remote server. The patient profile may be retrieved from the remote server by the respiratory apparatus. The profile may include acclimatisation parameters. The patient may be able to specify and store one or more acclimatisation parameters, such as a time period/ramp rate for humidity/temperature. The time period/ramp rate and/or other acclimatisation parameters might be predefined. The respiratory apparatus could apply these acclimatisation parameters when a user triggers acclimatisation process.

Acclimatisation is particularly useful for a homecare patient that would be using respiratory apparatus 10 for a long time and/or without assistance. A homecare patient uses the respiratory apparatus 10 in a home environment, as opposed to (or in conjunction with) a hospital or medical facility. The acclimatisation preference settings selected by the patient and/or the controller can track the settings selected by the patient and if there is a common setting selected, then the controller may determine that as a preferred setting. The preferred setting would be stored.

Various non-limiting examples that implement the example acclimatisation methods, described above will now be described.

A first example will be described with reference to FIG. 4. The apparatus of FIG. 1 is configured to implement the acclimatisation method of FIG. 4 (in addition to other typical operations, step 30). This results in one example apparatus and method. The example relates to flow therapy by way of example, but the method/apparatus is not limited to this. In this example, humidity is varied. Dew point is the parameter used to indicate humidity, but as discussed that is one option only and the same acclimatisation could be implemented using any other humidity parameter.

First, step 30, the respiratory apparatus 10 provides flow therapy in the usual way. It monitors also for a trigger, step 31 (either patient or apparatus initiated). If a trigger is received the method activates the acclimatisation method. For example, a user presses the comfort button or provides an input for improved comfort via the UI 45. The trigger may be initiated manually if the user is uncomfortable. Alternatively the trigger is initiated by the respiratory apparatus 10 if the interface is detected as being removed above a threshold value. The interface 51 being removed more than a threshold is indicative that a patient is struggling to become comfortable and adhere to the flow therapy. Therefore the controller 19 automatically trigger acclimatisation (or offer the option to trigger acclimatisation—see FIG. 3) if the interface 51 is considered as being removed more than a threshold number of times. In this example, acclimatisation comprises a humidity ramp from an initial comfort level to an operational level that allows the user to become more acclimatised to the humidity at the particular flow rate.

The controller 19 then takes the acclimatisation action 88 of reducing humidity using closed loop control to control dew point at the outlet of the water chamber. To do this, the controller 19 is configured to determine, step 32, a lower dew point set point (initial comfort level humidity gas flow parameter) e.g. the new dew point may be between about 30% to about 75% lower than current dew point. Optionally between about 50% to about 65% lower. The new dew point set point is optionally at least about 3 deg C lower than current set point. Optionally at least 5 about deg C lower. In one example the dew point is reduced by about 10 deg C, for example from about 37 deg C to about 27 deg C. The lowest dew point set point limit is about 25 deg C. Defined another way, the heater plate power is reduced by between about 10% to about 30%.

Next, under closed loop control to meet the dew point, the controller 19 reduces power provided to the heater plate 59, step 33. For example, to reduce power, the duty cycle to the heater plate 59 is reduced; or power to heater plate 59 is switched off altogether; or alternatively voltage or current to the heater plate 59 is switched off. Optionally the heater plate power is switched off in order to reduce the dew point as quickly as possible. The heater wire 58 power may be maintained at the same limit. The feedback is based on the EOH (end of hose) temperature sensor 14 that still uses 37 deg C as the set point. This ensures that there is no/reduced condensate.

The temperature at the water chamber outlet is measured using a chamber outlet temperature sensor 53C. The heater plate 59 remains at reduced power until the chamber outlet measures the lower dew point set point (e.g. is chamber outlet temp measured to be lower temperature-that is, the initial comfort level).

Next, the controller 19 takes the acclimatisation action 88 of increasing humidity back to the operational level, step 34. The controller is configured to control the heater plate to ramp to the operational humidity level. It does this by ramping the dew point from the lower set point (initial comfort level) back to the initial user set point (operational level). Under closed loop control, the controller 19 is configured to control the power to the heater plate 59 such that the dew point temp increases from about the initial comfort level 25 deg C to the operational level of about 37 deg C over a time period (or at a predetermined ramp rate) . For example the time period may be between 15 min and 1 hour, and optionally between 30 min and 45 min. The humidity ramp can be implemented by ramping the dew point i.e. the chamber outlet temperature. Alternatively, the heater plate power duty cycle may be ramped up over the set time e.g. from 10% to 80% over the time period to ramp the humidity output of the chamber.

After the operational level is reached, step 34, then normal operation is provided by the respiratory apparatus at operational levels, step 30.

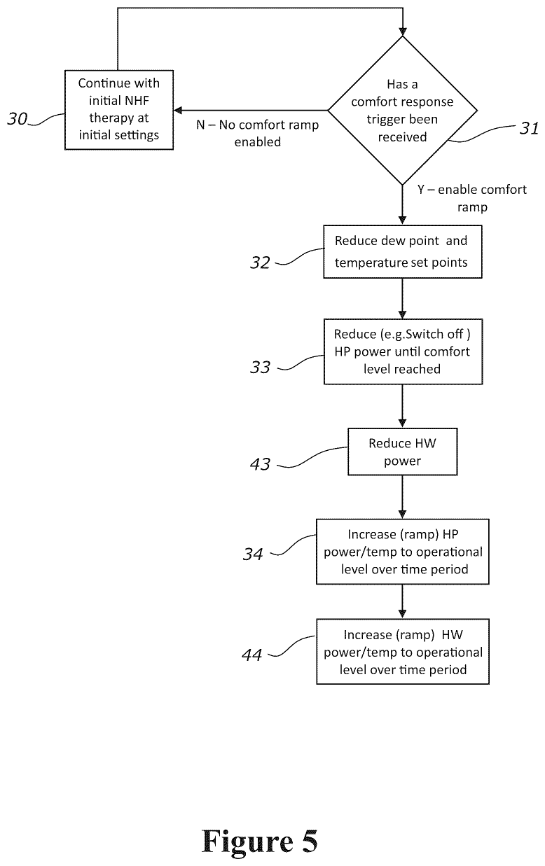

A second example will be described with reference to FIG. 5. The apparatus of FIG. 1 is configured to implement the acclimatisation method of FIG. 5 (in addition to other typical operations, step 30). This results in one exemplary example of an apparatus and method. The example relates to flow therapy by way of example, but the method/apparatus is not limited to this. In this example, humidity is varied. Dew point is the parameter used to indicate humidity, but as discussed that is one option only and the same acclimatisation could be implemented using any other humidity parameter. In this example both the humidity and temperature are varied through the heater plate 59 and the heater wire 58.

First, step 30, the respiratory apparatus 10 provides flow therapy in the usual way. It monitors also for a trigger, step 31, (either patient or apparatus initiated) to active the acclimatisation process as described in the first example. The controller 19 then takes the acclimatisation action of reducing humidity such the same as described in steps 32, 33 of FIG. 4.

The controller 19 then takes the acclimatisation action 88 of reducing temperature. It does this by reducing the temperature set point (in this case at the patient end of hose “EOH”) to an initial comfort level, step 32, and under closed loop control reducing power to the heater wire 58 (heater wire), step 43 to achieve that temperature set point. The temperature set point based on the EOH temperature sensor 14 is reduced to a lower temperature. The EOH temperature is reduced by the same “amount” as the chamber outlet set point e.g. by about 30% to about 75% lower. Optionally, the EOH temperature is always higher than the chamber outlet temperature set point e.g. by 3 deg C. Temperature at the chamber outlet is measured using a chamber outlet temperature sensor 53C. The heater plate power remains at reduced power until the chamber outlet measures the lower dew point set point (e.g. 25 deg C). The heater wire 58 power is reduced. The temperature at the EOH is also monitored to ensure that it remains above the chamber outlet temperature.

Next, the controller 19 takes the acclimatisation action of increasing humidity back to the operational level, step 34 as described for FIG. 4. The controller 19 also takes the acclimatisation action 88 of increasing temperature back to the EOH set point (operational level), step 34. The acclimatisation action 88 implementation is to control the heater wire 58. It does this by ramping the set point temperature at EOH from the initial comfort level to the operational level. The controller 19 is configured under closed loop control to control the heater wire 58 power so it ramps over the same time period to the operational level EOH set point. EOH set point is maintained at 3 deg above the chamber outlet temp set point. Alternatively, the ramp time period is shorter than the time period for the humidity ramp i.e. rate of ramp is faster than the HP power ramp. For example, the heater wire ramp time period may be 50% faster than the HP ramp time. For example the heater wire power may be ramped such that it reaches temperature set point (operational level) in about 7 min to about 30 min. The heater wire temperature ramp is not greater than the heater plate ramp to avoid formation of condensation.

A third example will be described with reference to FIG. 5. The apparatus of FIG. 1 is configured to implement the acclimatisation method of FIG. 5 (in addition to other typical operations, step 30). This results in one example of an apparatus and method. The example relates to flow therapy, but the method/apparatus is not limited to this. In this example, humidity is varied. Dew point is the parameter used to indicate humidity, but as discussed that is one option only and the same acclimatisation could be implemented using any other humidity parameter. In this example both the humidity and temperature are varied through the heater plate 59 and the heater wire 58.

This example is similar to the first or second example, except that no trigger is received during operation. Rather, the acclimatisation is activated on start-up of the respiratory apparatus 10-either automatically or upon receiving a manual trigger. This means that the acclimatisation actions of reduction of humidity and/or temperature is not required. Rather, only acclimatisation actions 88 of gradual (ramp) of humidity and/or temperature are required. The controller 19 implements a humidity and/or temperature ramp action at start-up. At start up the user would select a dew point set point e.g. 37 deg C dew point.

Alternatively, this acclimatisation could be triggered manually or automatically after an initial warm up. The water may be heated until the heater plate 59 temperature reaches a threshold. During warm up the heater plate 59 is heated as fast as possible to a target heater plate temperature. Gas flow may not be activated until the heater plate 59 is warmed up. Alternatively, the flow rate is started the heater plate 59 is heated as fast as possible to a warm-up set point. The warm-up set point relates to a heater plate temperature. A further form, the warm-up set point temp may relate to a temperature of gases as measured at the chamber outlet with a chamber outlet temp sensor.

The acclimatisation is optionally manually initiated by a user after a warm-up period is completed. If a patient does not initiate the acclimatisation method for a set time, then the heater plate 59 is heated as quickly as possible to achieve a gases temperature of the set dew point. This is measured at the chamber outlet or near the chamber outlet with chamber outlet temp sensor 53C.

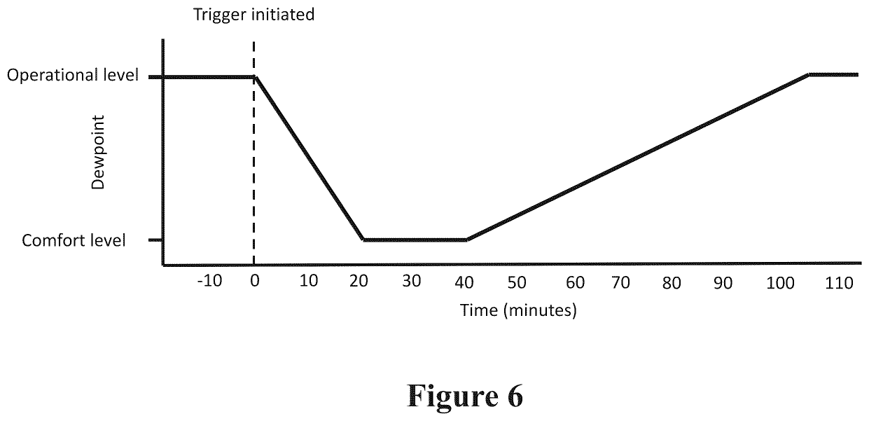

FIG. 6 shows an example pattern for the change in humidity during an acclimatisation period. Prior to receiving the trigger the respiratory apparatus is operating at an operating level. For example, this may be a dew point of 37 degrees. At time 0 a trigger is activated, and shortly afterwards the system reduces the humidity and temperature to reach a comfort level. The reduction is shown as a linear ramp over a time period. The time period of the example if 20 minutes, although other times are possible. In other cases a plurality of different ramps may be used in consecutive acclimatisation actions to reach the comfort mode., The comfort level may be held for a level of time, such as 5 or 10 minutes, or 20 minutes as shown. After sufficient operation or time at the comfort level acclimatisation actions are used to return the humidity and temperature to the original operational level. Again, a linear or constant ramp is shown between the comfort level and operational level. The time period of the increase in humidity back to the operational level may be longer, or shorter, than the time period of the reduction to the comfort level. A longer time period may improve a patient's adherence or adaption to the operational level.

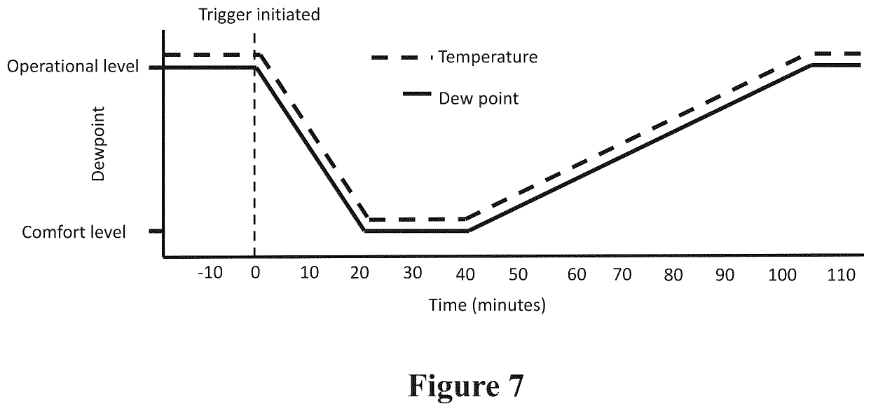

FIG. 7 shows an example pattern for the change in temperature and humidity during an acclimatisation method. The dew point curve shown in similar to FIG. 6, however a temperature of the gases flow is now also decreased from an operational level to a comfort level. The temperature is shown as decreasing concurrently with the humidity, and at the same rate. However, in other examples the temperature and humidity may decrease separately or at faster and/or slower rates than each other.

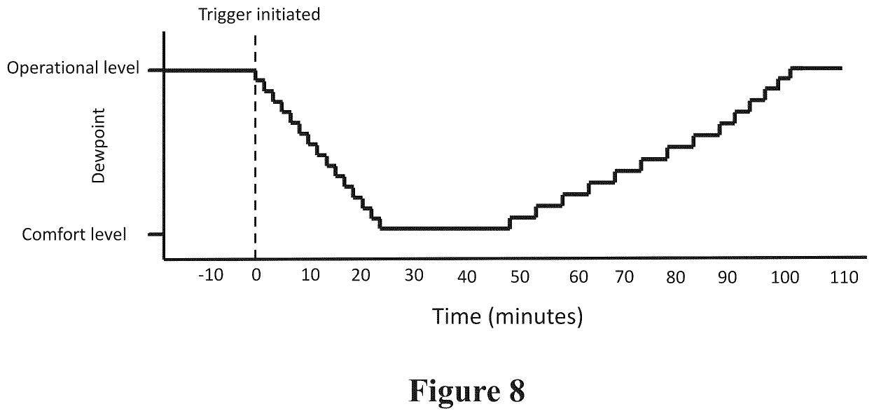

FIG. 8 shows example pattern for the change in humidity during an acclimatisation method. FIG. 8 shows a stepped humidity change where the humidity is allowed to drop by a selected or pre-set amount each step and pause before dropping again. FIG. 8 shows a constant step size during the decrease from operational level to comfort level. However, the step size may be varied either due to patient preference or because of system constraints. During the increase back to operational level a first period has a relatively slow increase (large step) with a second period having a faster increase (small step). As shown the shorter step may be used closer to the operational level where the patient has adapted to the changes. Alternatively the shorter step may be used closer to the comfort level, where the patient is less likely to feel uncomfortable. In some cases more than one or two periods with different step sizes may be used, or the step size may adjust, for example in real-time, based on patient or system feedback.

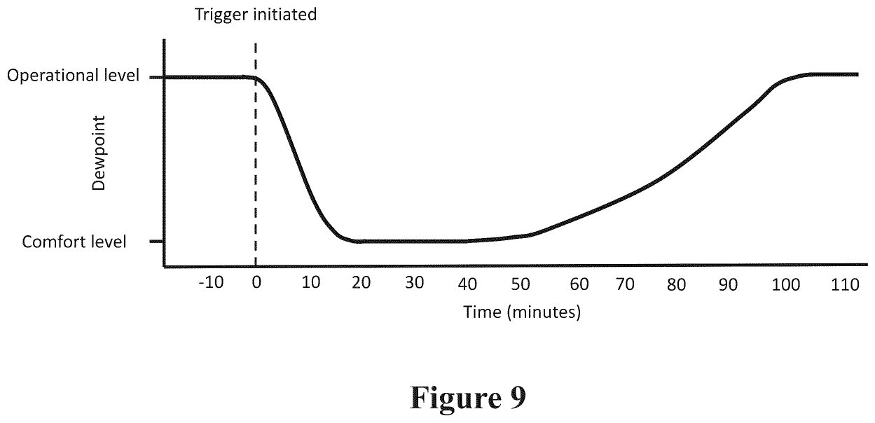

FIG. 9 shows an example pattern for the change in humidity during an acclimatisation period. FIG. 9 shows a non-linear change in humidity, although the overall profile follows the linear change as shown in FIG. 6. This may not be the case. For example, in some cases there may be oscillations or step-backs at the comfort level or during the increase to the operational level to improve patient comfort, or better enable the patient to adapt to the increase to the operational level.

The acclimatisation method may be applied to other respiratory apparatus 10 or systems. FIG. 1 shows a heated pass-over humidifier. A heated pass-over humidifier typically contains a volume of water that reduces as water is evaporated. The entirety of the water reserve is heated to achieve humidification of the patient-bound airstream. In some cases a heated pass-over humidifier comprises a float. The float in the water chamber 52. The float operates a valve to release water (or other liquid(s) used in the water chamber 52) from a water reservoir into the chamber 52 when the water level drops below a threshold. This may be configured such that a substantially constant water level is maintained and/or so that the water level in the chamber is maintained above a threshold level.

A further humidification system is a vaporization humidifier. For example, WO2016036260A1, incorporated herein by reference, introduces a deterministically controlled humidification system (also referred to as a vaporization humidifier). A vaporization humidifier typically applied a portion of water (or suitable liquid) onto a heating surface. The portion of water may be controlled or dosed. Depositing the water on the heating surface causes it to be vaporized into a gases flow. The humidified gases flow can then be provided to the patient Vaporization humidifiers may be controlled in a deterministic manner wherein the portion comprises a determined quantity of liquid. The determined quantity of liquid configured to achieve the target humidity. In some cases a controller is configured to determine, or look-up, the quantity of liquid required. In a vaporization humidifier water flow may be controlled to achieve a target humidity, whereas, in a float humidifier water flow is controlled to maintain a volume of water in the chamber.

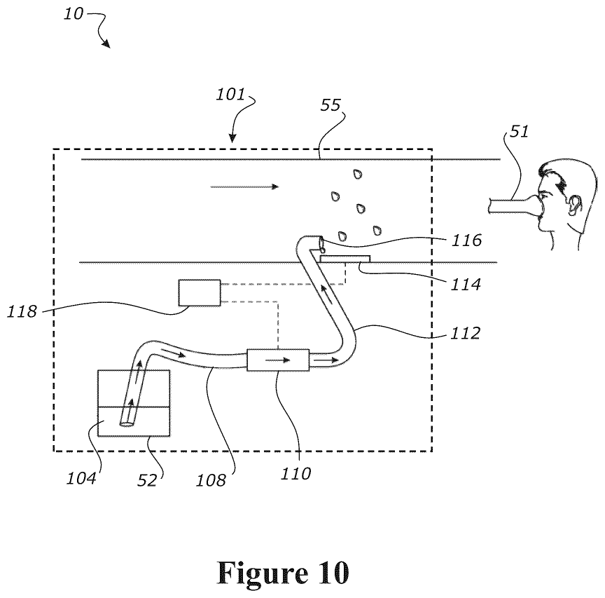

FIG. 10 shows an example respiratory apparatus 10 comprising a vaporisation humidifier form of a respiratory humidification system 101. The respiratory humidification system 101 includes a conduit 102 (which may also be referred to herein as “a gases channel”, “a breathing tube,” or “an inspiratory tube”) adapted to receive gases from the flow generator (not shown) and/or another gases source and channel the gases to an outlet, such as a patient interface 51. In use, gases typically flow from the flow generator to the respiratory humidification system 101 (for example, through the conduit 102), and from the respiratory humidification system 101 to the outlet or patient interface 51 (for example, through the conduit 102) in a downstream direction.

As shown in FIG. 10 a non-limiting exemplary configuration respiratory humidification system 101 includes a fluid reservoir or water chamber 52 which in use houses a fluid 104. “Fluid” in this context may refer to liquids or fluent solids suitable for humidifying respiratory gases and may include, for example, water. The fluid 104 may be a water with additives that are more volatile than water. The water chamber 52 is fluidly or otherwise physically linked to a meter or metering arrangement (also referred to as a liquid flow controller or water flow controller herein) 110. The metering arrangement 110 is configured to meter fluid from the fluid reservoir 106 to a heater plate 114, or other system configured to provide a heating surface. The metering arrangement 110 can further include a pump. The pump can be a positive displacement pump, such as, for example, a piezoelectric diaphragm pump, a peristaltic pump, a micro-pump, or a progressive cavity pump. The pump can also be a pressure feed, such as a gravity feed in series with a control valve. The metering arrangement 110 can include a wicking structure that employs capillary action to controllably meter the water to the wicking element and/or to the heating surface.

A component of the respiratory apparatus 10 or of the respiratory humidification system 101 can include a controller 118 that can control the operation of components of the respiratory apparatus 10 or of the respiratory humidification system 101, including but not limited to the flow generator, the metering arrangement 110, and/or the heater plate 114. he first and second fluid conduits 108, 112 may be configured to communicate fluids to various components of the respiratory humidification system 101. As illustrated in FIG. 10, a first fluid conduit 108 may be configured to fluidly communicate fluid from the fluid reservoir 52 to the metering arrangement 110, and the second fluid conduit 112 may be configured to fluidly communicate fluid from the metering arrangement 110 to the humidification housing conduit 55 or humidification housing. The second fluid conduit may have an outlet 116 configured to direct the liquid onto the heater plate 114. It will be understood that different arrangements are possible, where the system still allows the vaporisation of a controlled amount of liquid.

Metering arrangement 110 can be controlled by a water flow controller 118 (water flow controller may form, or be combined with, controller 19 and/or a humidifier controller). The metering arrangement 110 may comprise a pump in an open-loop configuration. The metering arrangement 110 may comprise a pump or a flow actuator in series with a flow sensor in a closed-loop configuration. In other configurations, a pump or a flow actuator in series with a flow sensor in a closed-loop configuration can be used. The water flow controller may configure the metering arrangement to provide a continuous flow of water in the range of 0 mL/min to approximately 10 mL/min. The metering system 110, may be configured to ensure that the surface of the heater plate 114 is entirely wetted (saturated). A fully wetted surface may allow for improved deterministic control of the humidity. A wetted surface also means that humidity can be increased more quickly as water travels more quickly over a wet surface than it does over a dry surface.

The heater plate 114 can have a wicking element configured to distribute the metered fluid to the heater plate 114. In some configurations, the wicking element is configured to wick the metered fluid evenly across the surface of the heater plate 114. The heater plate 114 may be configured to vaporize the metered fluid such that it becomes entrained in the gases flow in use by the respiratory therapy system 10. The heater plate 114 can be configured to be maintain a heating surface at a temperature range. The temperature range may be between approximately 30 °C and approximately 99.9 °C.

The metering arrangement 110 may be configured to meter or allocate fluid to the humidifier and/or to the heater plate 114 at metering rates that raise the moisture content of gases passing through the conduit 55 such that the gases reach a predetermined, calculated, or estimated humidity level representing a level of gases humidification needed or desired by a patient while taking care to reduce or eliminate the likelihood of undue moisture accumulation in the gases channel 102. To control the humidification, in one example, the controller 118 can control the metering rate of the metering arrangement 110 based on any one or more of:
a measured flow rate of gases passing through the conduit 55,
a measured moisture value corresponding to the humidity of gases upstream of the humidification housing,
a measured pressure level corresponding to the pressure level in the gases channel 102, or
a combination thereof.

The controller 118 can control the metering rate of the metering arrangement 110 based on a combination of one or more of measured inputs such as
measure flow rate of gases passing through the conduit 55;
a measured moisture value corresponding to the humidity of gases upstream of the humidification housing,
a measured flow rate of gases passing through the conduit 55 and
a measured pressure level corresponding to the pressure level in the conduit 55.

The respiratory therapy system 10 or the respiratory humidification system 101 may include deterministic or open loop control with various control systems possible. In general, deterministic control may allow for on-demand humidification achieved by controlling certain input variables, for example, by controlling water flow to the heating surface. In some configurations, control of the water flow rate to the heating surface may be based on a flow rate of the gases in the gases channel. Control of the water flow rate to the heating surface may be based on an evaporation rate of the water from the heating surface. Control of the water flow rate to the heating surface may be based on a temperature of the heating surface wherein the temperature of the heating surface is maintained at a constant temperature. Control of the water flow rate to the heating surface may be based on a temperature of the heating surface wherein the temperature of the heating surface is controlled. Control of the water flow rate to the heating surface may be based on an absolute or barometric pressure of the gases at or near the inlet location. Control of the water flow rate to the heating surface may be based on a dew point temperature of the gases at the inlet location. Control of the water flow rate to the heating surface may be based on an enthalpy provided by the heating surface. Control of the water flow rate to the heating surface may be based on a power level provided by the heating surface. Control of the water flow rate to the heating surface may be based on a temperature of the gases at the inlet location. The dew point temperature of the gases at the inlet location may be derived by processing information provided by a temperature sensor and a humidity sensor. Control of the water flow rate to the heating surface may be based on the dew point temperature of the gases at the inlet location. Control of the water flow rate to the heating surface may be based on a relative humidity level of the gases at the inlet location. Control of the water flow rate to the heating surface may be based on an effective heating area of the heating surface. Control of the water flow rate to the heating surface may be based on a pressure level of the gases in the gases channel. Control of the water flow rate to the heating surface may be based on a velocity of the gases flowing in the gases channel. Control of the water flow rate to the heating surface may be based on a temperature of the water flow. As shown and described in reference to Figure IE below, the respiratory therapy system 100 and/or the components thereof (including the respiratory humidification system 101) may include a number of sensors to measure these variables.

The system may have various control systems and configurations to provide the described humidification. For example the control system may comprise a humidification fluid flow control sub-system that monitors and controls the rate at which fluid is metered to the humidification region and, more specifically, to the heater plate 114. A fluid flow sensor measures the flow of the humidification fluid and provides the measurement to a fluid flow controller. The controller compares the measured fluid flow rate with the desired fluid flow rate (which may be predefined, estimated, or deterministically derived), and adjusts the power level to the metering arrangement accordingly.

In some cases a preheater may be used before the gas flow is provided to the heater plate 114. The inlet and pre-heating control sub-system of may measure the air and/or gas coming into the system using inlet sensors to determine the ambient humidity of the incoming gas flow rate and the incoming gas pressure. The gas flow may then be heated with a pre-heater. An inlet temperature sensor downstream of the pre-heater can then measure the temperature of the heated gas, which can be compared with a calculated temperature set or defined by the controller, with signals sent to the pre-heater to adjust the temperature accordingly.

In some cases a water flow control subsystem may be used. In one example water enters a water pump from a water source. The water pump may pump the water into the respiratory apparatus 10. A water flow sensor is positioned downstream from the pump and measures the flow rate of the water which is output to a liquid flow controller. The liquid flow controller provides a feedback loop whereby the water pump is adjusted based on a comparison of and a calculated water flow rate. The calculated water flow rate is determined by the overall system controller.

In some cases a heated surface control subsystem may be used. In some cases the heater plate 114 comprises multiple heater plates, or two heating zones are located on one heater plate. The heated surface controller acts when water flow and gas flow, are routed over the heater plate 114 or heating surface. The surface may include one or more surface temperature sensors which provide measurements of the surface temperature to a surface temperature controller. The surface temperature controller provides a feedback and control mechanism whereby heater plate 114, which either forms the surface or is in thermal communication with the surface, is adjusted. The surface temperature controller may compare the sensed temperature to a calculated surface temperature. The calculated surface temperature is determined by the overall system controller of the humidifier or system. The overall system controller of the humidifier or respiratory apparatus may receive input sensors, including ambient humidity, incoming gas flow rate, incoming gas pressure and a set humidity (such as a dew point temperature). Optionally the overall system may also receive power inputs, such as the power delivered to the surface, or power delivered to the air. These, or further inputs may be used to calculate the set temperatures or mass flow rates discussed above.

In some cases, a deterministic humidifier has the advantage that a relatively small amount of water contacts the heater plate 114 at a given time. Therefore, if a change in humidity generation is required, the thermal resistance of the water is low, so the heater plate can change temperature faster and thus reduce/increase humidity faster than a heated pass-over humidifier.

The acclimatisation method may be used with a deterministic humidifier arrangement. In some cases, this is advantageous because the method can take advantage of the quicker changes in operational levels available to more quickly acclimatise a patient, or at least more quickly move between levels. Where the controller knows, for example, the flow rate and humidity of the gas, it can calculate the amount of water vapor to add to achieve the comfort dew point and thus perform acclimatisation actions 88 to reach the comfort target and return to the operational level. The acclimatisation method may also take advantage of further operational parameters of the deterministic humidifier, such as flow rates to perform the acclimatisation actions.

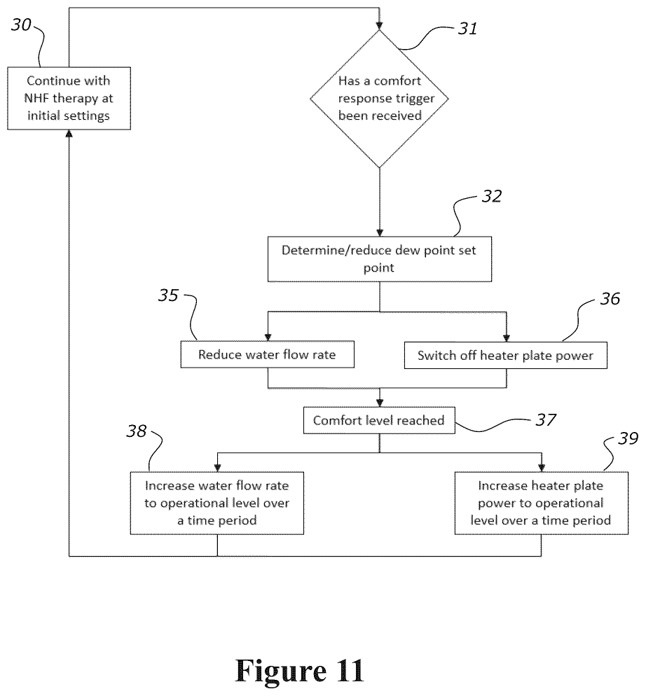

A further example will be described with reference to FIG. 11. The apparatus of FIG. 10 is configured to implement the acclimatisation method of FIG. 11 (in addition to other typical operations, step 30). The example relates to flow therapy by way of example, but the method/apparatus is not limited to this. In this example, humidity is varied by control of one or more of water flow rate and heater plate power. Dew point is the parameter used to indicate humidity, but as discussed that is one option only and the same acclimatisation could be implemented using any other humidity parameter.

First, step 30, the respiratory apparatus 10 provides flow therapy in the usual way. It monitors also for a trigger, step 31 (either patient or apparatus initiated). If a trigger is received the method activates the acclimatisation method, as described previously. In this example, acclimatisation comprises a humidity ramp from an initial comfort level to an operational level that allows the user to become more acclimatised to the humidity at the particular flow rate. The controller 19, 118 then takes the acclimatisation action 88 of reducing humidity using closed loop control to control dew point at the outlet of the water chamber. To perform the acclimatisation, the controller 19, 118 is configured to determine, step 32, a lower dew point set point (initial comfort level humidity gas flow parameter) e.g. the new dew point may be between about 30% to about 75% lower than current dew point, or other values as previously mentioned.

Next, the controller 19, 118 determines how to take the acclimatisation actions. This may comprise one or more of:
reducing the water flow rate, step 35;
turning of heater plate 114, step 36, and
reducing power provided to the heater plate 114, step 36.
   For example, to reduce power, the duty cycle to the heater plate 59 is reduced; or power to heater plate 59 is switched off altogether; or alternatively voltage or current to the heater plate 59 is switched off. Optionally the heater plate power is switched off in order to reduce the dew point as quickly as possible. The heater wire 58 power may be maintained at the same limit. The feedback is based on the EOH (end of hose) temperature sensor 14 that still uses 37 deg C as the set point. This ensures that there is no/reduced condensate in the conduit 55. The water flow rate can be controlled, for example by metering arrangement 110. The water flow rate could be controlled independently or in conjunction with a change in flow rate and/or temperature. The suitable water flow rate to reach the comfort level or perform an acclimatisation action may be determined based on a gas flow rate model. The water flow rate may be increase or decreased to ensure patient comfort, or to encourage adaption to the operational level. The acclimatisation actions 88 continue until the comfort level is reached, step 37. For example, to reduce humidity the water flow rate on to the heater plate 114 may be reduced, less water present on the heater plate reduces the amount of water evaporated into the gases flow, and therefore the humidity. In another example the temperature of the heater plate 114 may also be controlled.

Next, the controller 19, 118 takes the acclimatisation action 88 of increasing respiratory apparatus 10 back to the operational level, step 34. The controller 19, 118, is configured to control one or more of the heater plate 114 and the metering arrangement 110 to ramp to the operational level of water flow rate 38 and/or heater plate power (temperature) 39. It does this by ramping the dew point from the lower set point (initial comfort level) back to the initial user set point (operational level). For example, the controller 118 may be configured to control the power to the heater plate 114, using the control methods described above or otherwise, such that the dew point temp increases from an example initial comfort level 25 deg C to the operational level of about 37 deg Centigrade over a time period (or at a predetermined ramp rate) . The water flow rate may be increased in conjunction with the temperature. In some cases the increases may be offset, or taken in turns, to reduce the effective change in operating level for the patient. For example the time period may be between 15 min and 1 hour, and optionally between 30 min and 45 min. After the operational level is reached then normal operation is provided by the respiratory apparatus at operational levels, step 30.

In some cases, the use of a deterministic humidifier system encourages the control of water flow rate in addition to the heater plate power. In either case further parameters (for example, gases flow rate and/or pressure) may also be controlled. The use of a deterministic humidifier system (or other suitable humidifier system) may allow the rate of change from operational to comfort level (or vice-versa) to be increased or controlled more exactly. This is because the humidification process can be more precisely controlled using the metering apparatus 110. In some cases the time frame of the transition to the comfort level, or between the comfort level and the operational level (or between the operational level and comfort level) can be controlled more precisely. For example, a transition may be controlled over a predetermined period. In some cases the patient comfort can be monitored during a transition (e.g., by tracking the trigger of a further comfort response, or by monitoring mask removal) and the transition may be modified to increase comfort, for example by slowing the transition.

In some case a transition, for example from an operational level to a comfort level, comprises the water flow rate being reduced to the level required for the comfort level. In some cases the heater plate 114 power is reduced. The power may be reduced simultaneously with the water flow rate, or before or after the water flow rate. In one example the time for a transition (i.e., an acclimatisation action) reducing from a 38 degrees dewpoint to 34 degrees (flow constant) is approximately 2 minutes. Example transitions between the operational level and the comfort level may have a time period of up to 20 minutes, up to 10 minutes, about 2 minutes or about 5 minutes. In some case, for example to reduce the chance of an over-enthalpy condition, the transition period is about 10 minutes. In some cases, the transition period may be between 5 and 20 minutes, between 5 and 15 minutes or between 5 and 10 minutes. One or more of the control or acclimatisation methods described above can be applied to different humidification systems, such as a deterministic humidifier. These alternative humidifiers may have additional operational parameters to be controlled, such as water flow rate.

The acclimatisation method has been described with a concentration of controlling the humidity and/or temperature of the respiratory device. However, the method and system described herein may be applied to any of the operational parameters, if desired. For example one or more of humidity and/or temperature, flow rate, pressure and gases concentration. For example, if a patient is not used to a high flow rate, or the addition (or reduction) of supplementary oxygen, the system may increase or decrease the flow rate or supplementary oxygen rate between a comfort level and an operational level. This may help to improve patient comfort, and therefore adherence.

In some cases humidity and flow may be ramped, so as to acclimatise a patient to both a higher humidity and greater flow of gases in the system. This may ease a transition between a change in the delivery of gases to a patient based on a change of therapy. In another example the flow and/or pressure may be ramped independently of humidity, for example. In some cases the flow rate is ramped or modified along with another operational parameter. This may assist the feeling to the patient. For example, higher flow rates normally require higher humidities, by increasing these parameters together (either simultaneously or in turn) the increase towards the operational level may be made more comfortable.

One or more of the follow advantages might be achieved. The acclimatisation method acclimatises the patient to an operational parameter (e.g. humidity and/or temperature) from an initial comfort level to an operational level over a time period. As the parameter gradually increases, the patient tolerates each increase (acclimatises to each level) so experiences comfort for the acclimatisation period. By the time the operational level is reached, the patient can tolerate that operation parameter, even if they could not have done so at the outset. Because the patient can tolerate the operational level of the operational parameter, they are more likely to use the respiratory apparatus as directed and more likely to be compliant with the therapy provided by the respiratory apparatus.

A typical use might be that a patient uses a respiratory apparatus and gas flows are provided with operation parameters (e.g. humidity and/or temperature) at an operational level. The patient might find this operational level of the parameters uncomfortable, so the acclimatisation method is activated to drop the parameters to an initial comfort level which is tolerable, and then ramp up to the operational level, at which point the patient tolerates the operational level so that usual operation can continue. But other uses are possible, and this acclimatisation method can be used at any suitable time.

Some examples outcomes are:
Improved comfort to the patient for flow therapy when the patient is uncomfortable due to the flow of heated and humidified gases, particularly the gases being fully saturated. The gases optionally are fully saturated in order to make high flow comfortable and tolerable.
A user becomes acclimatised to the flow therapy that has a flow of gases with the gases being fully saturated.
Improved compliance to the therapy by the user as the user can become acclimatised.
The user has control that allows the user to initiate the acclimatisation at any point. This will improve compliance since the user has control on the humidity and/or temperature provided.
Determination that the patient is not complying e.g. removing the patient interface and then automatically initiating the acclimatisation actions (e.g., a humidity ramp). The acclimatisation actions should help to improve compliance by the patient.
A flow rate may be maintained at the set point and therefore flow therapy mechanisms continue even with lower humidity.
Acclimatisation can be useful when a patient is using high flow therapy while sleeping. This acclimatisation method potentially could be activated on before sleeping and may help a patient to be compliant as the patient is entering a sleep state.

Aspects of the controller and methods described above may be operable or implemented on any type of specific-purpose or special computer, or any machine or computer or server or electronic device with a microprocessor, processor, microcontroller, programmable controller, or the like, or a cloud-based platform or other network of processors and/or servers, whether local or remote, or any combination of such devices.

The controller described in connection with the examples disclosed herein may be implemented or performed with a general purpose processor, a digital signal processor (DSP), an application specific integrated circuit (ASIC), a field programmable gate array (FPGA) or other programmable logic component, discrete gate or transistor logic, discrete hardware components, or any combination thereof designed to perform the functions described herein. A general purpose processor may be a microprocessor, but in the alternative, the processor may be any conventional processor, controller, microcontroller, circuit, and/or state machine. A processor may also be implemented as a combination of computing components, e.g., a combination of a DSP and a microprocessor, a number of microprocessors, one or more microprocessors in conjunction with a DSP core, or any other such configuration.

The methods or algorithms described in connection with the examples disclosed herein may be embodied directly in hardware, in a software module executable by a processor, or in a combination of both, in the form of processing unit, programming instructions, or other directions, and may be contained in a single device or distributed across multiple devices. A software module may reside a storage medium such as RAM memory, flash memory, ROM memory, EPROM memory, EEPROM memory, registers, hard disk, a removable disk, a CD-ROM, or any other form of storage medium known in the art. A storage medium may be coupled to the processor such that the processor can read information from, and write information to, the storage medium. In the alternative, the storage medium may be integral to the processor.

In its various aspects, embodiments of the disclosure can be embodied in a computer-implemented process, a machine (such as an electronic device, or a general purpose computer or other device that provides a platform on which computer programs can be executed), processes performed by these machines, or an article of manufacture. Such articles can include a computer program product or digital information product in which a computer readable storage medium containing computer program instructions or computer readable data stored thereon, and processes and machines that create and use these articles of manufacture.

1. A respiratory apparatus comprising:
a flow generator for providing a flow of gas
a humidifier for humidifying the flow of gas, and
a controller configured to operate the breathing apparatus for acclimatisation by:
reducing one or more of a humidity and a temperature of the flow of gas from an operational level to a comfort level, and
over a first time period, increasing one or more of the humidity and the temperature of the flow of gas from the comfort level to the operational level.

2. A respiratory apparatus according to claim 1 wherein prior to reducing one or more of the humidity and the temperature of the flow of gas, one or more of the humidity and temperature of the flow of gas is at an operational level.

3. A respiratory apparatus according to claim 1 or 2 wherein increasing one or more of the humidity and temperature of the flow of gas comprises ramping one or more of the humidity and temperature over the first time period.

4. A respiratory apparatus according to any preceding claim wherein a duration of the first time period is determined prior to reducing the humidity and/or temperature of the flow of gas.

5. A respiratory apparatus according to any preceding claim wherein a duration of the first time period is:
at least about 10 minutes,
at least about 15 mins,
at least about 30 minutes,
between about 30 minutes and about 40 minutes,
about 35 minutes,
at least about 40 minutes
about 45 minutes
between about 30 minutes and about 50 minutes,
between about 30 minutes and about 60 minutes
between about 35 minutes to about 1 hour,
at least about 1 hour,
between about 1 hour and about 2 hours, or
up to about 2 hours
calculated from a rate of about 1 degree C/5 minutes, to ramp from initial comfort level to the operational level (e.g. 31 degrees dew point to 37 degree C dewpoint would take about 30 minutes)

6. A respiratory apparatus according to any preceding claim wherein prior to reducing one or more of the humidity and temperature of the flow of gas, one or more of the humidity and temperature of the flow of gas is at an operational level for:
at least about 10 minutes,
at least about 15 mins,
at least about 30 minutes,
between about 30 minutes and about 40 minutes,
about 35 minutes,
at least about 40 minutes
about 45 minutes
between about 30 minutes and about 50 minutes,
between about 30 minutes and about 60 minutes
between about 35 minutes to about 1 hour,
at least about 1 hour,
between about 1 hour and about 2 hours, or
up to about 2 hours.

7. A respiratory apparatus according to any preceding claim wherein the operational level of humidity is about 31, 34 or 37 degrees C dewpoint at 100% RH.

8. A respiratory apparatus according to any preceding claim wherein the comfort level comprises a humidity three degrees below operational dew point level.

9. A respiratory apparatus according to any preceding claim wherein one or more of the humidity and temperature are reduced or increased by controlling the humidifier.

10. A respiratory apparatus according to any preceding claim wherein the humidifier comprises a heater plate configured to heat water to humidify the flow of gas.

11. A respiratory apparatus according to any preceding claim comprising a breathing conduit, the breathing conduit comprising a heater wire.

12. A respiratory apparatus according to claim 11 wherein the heater wire is coupled or couplable to an outlet of the humidifier.

13. A respiratory apparatus according to claims 10 to 12 wherein one or more of the humidity and temperature are reduced or increased by controlling one or more of the heater plate and heater wire.

14. A respiratory apparatus according to any of claims 1 to 13 wherein one or more of the humidity and the temperature of the flow of gases is sensed at one or more of:
an inlet of the humidifier;
in the humidifier;
an outlet of the humidifier;
a breathing conduit;
an inlet of the breathing conduit;
an outlet of the breathing conduit;
a patient interface;
an inlet of the patient interface; and
an outlet of the patient interface.

15. A respiratory apparatus according to any preceding claim wherein:
the humidity of the flow of gas is indicated by a humidity parameter, and
the temperature of the flow of gas is indicated by a temperature parameter.

16. A respiratory apparatus according to claim 15 wherein the humidity parameter is one or more of:
relative humidity;
absolute humidity; and
dew point.

17. A respiratory apparatus according to claim 15 or 16 wherein the temperature parameter is one or more of:
Temperature of gas flow;
Temperature of heater plate; and
Temperature of heater wire.

18. A respiratory apparatus according to any preceding claim further comprising one or more humidity and/or temperature sensors at one or more of
an inlet of the humidifier;
in the humidifier;
an outlet of the humidifier;
a breathing conduit;
an inlet of the breathing conduit;
an outlet of the breathing conduit;
a patient interface;
an inlet of the patient interface; and
an outlet of the patient interface.

19. A respiratory apparatus according to any preceding claim wherein the controller is configured to operate the breathing apparatus for patient comfort by upon one or more of:
receiving input from a user;
an internal trigger; and
start-up of the respiratory apparatus.

20. A respiratory apparatus according to any preceding claim comprising a metering apparatus configured to control a water flow rate in the humidifier

21. A respiratory apparatus according to claim 20 wherein one or more of the humidity and temperature are reduced or increased by controlling one or more of the heater plate, heater wire and metering apparatus.

22. A respiratory apparatus according to any preceding claim wherein the step of reducing one or more of a humidity and/or a temperature of the flow of gas from an operational level to a comfort level occurs over a second time period, optionally the second time period being up to 1 hour, up to 30 minutes, up to 20 minutes, between 5 and 15 minutes, or about 10 minutes or less than 5 minutes.

23. A respiratory apparatus according to any preceding claim wherein one or more of the humidity and temperature are ramped over the first time period, optionally the ramp being linear, stepped or non-linear.

24. A respiratory apparatus according to claim 23 wherein the ramp rate is predefined.

25. A respiratory apparatus according to any preceding claim wherein the humidifier comprises a heated pass-over humidifier, a heated pass-over humidifier with a float, or a vaporization humidifier.

26. A respiratory apparatus according to any preceding claim comprising the step of determining if a user has removed a patient interface and, based on determining removal of a patient interface, determining whether to operate the acclimatisation, optionally where determining whether to operate the acclimatisation comprises determining one or more of the number of times, or length of time the patient interface has been removed.

27. A respiratory apparatus according to any preceding claim wherein the comfort level comprises a dew point between 30% and 75% lower than the operational level.

28. A respiratory apparatus according to any preceding claim wherein one or more acclimatisation actions are used to increase or decrease one or more of the humidity and the temperature of the flow of gas.

29. A respiratory apparatus according to any preceding claim wherein the apparatus comprises a user interface, the user interface configured to allow the user to trigger acclimatisation, optionally wherein the user interface comprises a button or touch screen.

30. A respiratory apparatus according to claim 29 comprising a step of selecting each of the one or more acclimatisation actions based on a determination of current patient comfort, optionally patient comfort is determined by one or more of user interface removal and trigger of the acclimatisation button.

31. A respiratory apparatus according to any preceding claim wherein the controller is configured to operate the breathing apparatus for acclimatisation by:
reducing a flow rate of the flow of gas from the operational level to the comfort level, and
over a first time period, increasing the flow rate of the flow of gas from the comfort level to the operational level.

32. A respiratory apparatus according to claim 31 wherein reducing the flow rate and increasing the flow rate is simultaneous with reducing one or more of a humidity and a temperature and increasing one or more of a temperature respectively.

33. A respiratory apparatus according to claim 32 wherein reducing the flow rate and reducing one or more of a humidity and a temperature are performed separately.

34. A respiratory apparatus according to any one of claims 29 to 33 wherein the operational level is a high flow therapy level.
